(19) United States
(12) Patent Application Publication
(10) Pub. No.: US 2026/0029407 A1
(43) Pub. Date: Jan. 29, 2026

(54) METHODS FOR CHARACTERIZING A PROTEIN

(71) Applicant: Regeneron Pharmaceuticals, Inc., Tarrytown, NY (US)

(72) Inventors: Timothy-neil Tiambeng, Yonkers, NY (US); Yuetian Yan, Chappaqua, NY (US); Shunhai Wang, Scarsdale, NY (US)

(21) Appl. No.: 19/281,424

(22) Filed: Jul. 25, 2025

Related U.S. Application Data
(60) Provisional application No. 63/676,178, filed on Jul. 26, 2024.

(51) Int. Cl. G01N 33/68; B01D 15/34; C07K 14/005; C12N 15/86; G01N 30/32; G01N 30/72

(52) U.S. Cl. CPC G01N 33/6848; B01D 15/34; C07K 14/005; C12N 15/86; G01N 30/32; G01N 30/72; C12N 2750/14122; C12N 2750/14143; C12N 2750/14151; G01N 2030/324

(57) ABSTRACT

The present disclosure provides methods and systems for characterizing protein components of a viral capsid of a viral vector. In particular, the methods comprise subjecting a viral vector to size exclusion chromatography under a denaturing mobile phase to separate the protein components. Further, the present disclosure also provides methods for identifying and/or characterizing at least one low molecular weight (LMW) antibody species in a sample, wherein the method comprises subjecting the sample to size exclusion chromatography (SEC) column under a denaturing mobile phase to separate the LMW antibody species, and detecting the LMW antibody species by mass spectrometry to identify and/or characterize at least one LMW antibody species.

### CROSS REFERENCE TO RELATED APPLICATION

This application claims priority to, and the benefit of, U.S. Provisional Application No. 63/676,178, which was filed on Jul. 26, 2024, the disclosure of which is hereby incorporated by reference in its entirety.

### BACKGROUND

Viral vector products have emerged as pivotal delivery tools in clinical gene therapy. Adeno-associated virus (AAV) has received considerable attention as a highly effective viral vector for gene therapy due to its low immunogenicity and its ability to effectively infect a variety of cell and tissue types. Significant progress has been made in the last decade to adapt this viral system for use in human gene therapy.

AAVs are composed of three viral proteins, VP1, VP2, and VP3, which assemble into an icosahedral capsid that can encapsulate a single-stranded DNA payload containing a gene of interest. Characterization of AAV capsid protein attributes, such as serotype identity, capsid protein stoichiometry, and capsid post-translational modifications, are necessary to ensure product and process consistency.

Liquid chromatography-mass spectrometry (LC-MS) based approaches, such as hydrophilic interaction chromatography (HILIC) and reversed-phase liquid chromatography (RPLC), are commonly used for separation and characterization of intact viral protein (VP). However, these methods often use strong ion pairing agents, such as difluoroacetic acid (DFA) or trifluoroacetic acid (TFA), which are suboptimal for highly sensitive MS-based detection of AAV samples that are typically low concentration.

Therefore, it will be appreciated that a need exists for sensitive methods and systems for characterizing viral proteins of AAVs.

### SUMMARY

This disclosure provides methods for characterizing AAVs. In one exemplary embodiment, a method is provided for separating protein components of a viral capsid of a viral protein, the method comprising: obtaining a sample comprising the viral vector; and subjecting the sample to size exclusion chromatography under a denaturing mobile phase to separate the protein components of the viral capsid of the viral vector.

In one aspect, the viral vector is an adeno-associated virus (AAV) vector. In another aspect, the AAV vector comprises a serotype selected from a group consisting of AAV1, AAV2, AAV3, AAV4, AAV5, AAV6, AAV7, AAV8, AAV9, AAV10, AAV11, AAV12, variations thereof, and combinations thereof. In one aspect, the protein components of the viral capsid comprise VP1, VP2, and VP3 capsid proteins. In another aspect, the AAV vector is a recombinant AAV vector or an AAV vector encoding a heterologous transgene. In yet another aspect, the viral vector belongs to a viral family selected from the group consisting of Adenoviridae, Parvoviridae, Retroviridae, Baculoviridae, and Herpesviridae.

In one aspect, the denaturing mobile phase comprises acetonitrile, formic acid, and ammonium formate in water. In one aspect, the concentration of acetonitrile is about 30%. In another aspect, the concentration of formic acid is about 1%. In one aspect, the concentration of ammonium formate is about 5 mM. In another aspect, the concentration of ammonium formate is between about 5 mM to about 10 mM.

In one aspect, the flow rate of the mobile phase for SEC is between about 0.125 mL/min and about 0.2 mL/min. In another aspect, the flow rate of the mobile phase for SEC is about 0.2 mL/min. In one aspect, the column temperature for SEC is between about 50° C. to about 70° C. In another aspect, the column temperature for SEC is about 60° C.

In one aspect, fluorescence is detected using an excitation wavelength of 280 nm, and an emission wavelength of 348 nm.

In another exemplary embodiment, a method is provided for determining the stoichiometry of protein components of a viral capsid of a viral vector, the method comprising: subjecting a sample comprising the viral vector to size exclusion chromatography under a denaturing mobile phase to separate the protein components; and detecting the protein components by fluorescence to determine the relative abundance of the protein components separated by SEC, thereby determining the stoichiometry of the protein components of the viral capsid of the viral vector.

In one aspect, the viral vector is an adeno-associated virus (AAV) vector. In another aspect, the AAV vector comprises a serotype selected from a group consisting of AAV1, AAV2, AAV3, AAV4, AAV5, AAV6, AAV7, AAV8, AAV9, AAV10, AAV11, AAV12, variations thereof, and combinations thereof. In one aspect, the protein components of the viral capsid comprise VP1, VP2, and VP3 capsid proteins. In another aspect, the AAV vector is a recombinant AAV vector or an AAV vector encoding a heterologous transgene. In yet another aspect, the viral vector belongs to a viral family selected from the group consisting of Adenoviridae, Parvoviridae, Retroviridae, Baculoviridae, and Herpesviridae.

In one aspect, the denaturing mobile phase comprises acetonitrile, formic acid, and ammonium formate in water. In one aspect, the concentration of acetonitrile is about 30%. In another aspect, the concentration of formic acid is about 1%. In one aspect, the concentration of ammonium formate is about 5 mM. In another aspect, the concentration of ammonium formate is between about 5 mM to about 10 mM.

In one aspect, the flow rate of the mobile phase for SEC is between about 0.125 mL/min and about 0.2 mL/min. In another aspect, the flow rate of the mobile phase for SEC is about 0.2 mL/min. In one aspect, the column temperature for SEC is between about 50° C. to about 70° C. In another aspect, the column temperature for SEC is about 60° C.

In one aspect, the method further comprises subjecting the proteins components to mass spectrometry to identify the protein components. In one aspect, the SEC is coupled to the mass spectrometer. In one aspect, the mass spectrometer comprises a heated electrospray ionization source, an electrospray ionization source, a nano-electrospray ionization source, a microflow-nanospray electrospray ionization source, or a desorption electrospray ionization source. In another aspect, the mass spectrometer comprises a time-of-flight mass analyzer, a magnetic/electric sector mass analyzer, a quadrupole mass analyzer, or an orbitrap mass analyzer.

In one aspect, the method further comprises detecting the protein components by UV absorbance to determine the relative abundance of the protein components, wherein the UV detector is connected in tandem with the fluorescence detector.

In another exemplary embodiment, a method is provided for determining heterogeneity of protein components of a viral capsid of a viral vector, the method comprising: subjecting a sample comprising the viral vector to size exclusion chromatography under a denaturing mobile phase to separate the protein components; and subjecting the proteins components to mass spectrometry to determine the masses of the protein components, wherein the determined masses of the protein components are compared to theoretical masses to determine heterogeneity.

In another exemplary embodiment, a method is provided for determining heterogeneity of protein components of a viral capsid of a viral vector, the method comprising: subjecting a sample comprising the viral vector to size exclusion chromatography under a denaturing mobile phase to separate the protein components; and subjecting the proteins components to mass spectrometry to determine the masses of the protein components, wherein the determined masses of the protein components are compared to theoretical masses to determine heterogeneity.

In one aspect, heterogeneity comprises one or more of mixed serotypes, variant capsids, capsid protein amino acid substitutions, truncated capsid proteins, or modified capsid proteins.

In one aspect, the viral vector is an adeno-associated virus (AAV) vector. In another aspect, the AAV vector comprises a serotype selected from a group consisting of AAV1, AAV2, AAV3, AAV4, AAV5, AAV6, AAV7, AAV8, AAV9, AAV10, AAV11, AAV12, variations thereof, and combinations thereof. In one aspect, the protein components of the viral capsid comprise VP1, VP2, and VP3 capsid proteins. In another aspect, the AAV vector is a recombinant AAV vector or an AAV vector encoding a heterologous transgene. In yet another aspect, the viral vector belongs to a viral family selected from the group consisting of Adenoviridae, Parvoviridae, Retroviridae, Baculoviridae, and Herpesviridae.

In one aspect, the denaturing mobile phase comprises acetonitrile, formic acid, and ammonium formate in water. In one aspect, the concentration of acetonitrile is about 30%. In another aspect, the concentration of formic acid is about 1%. In one aspect, the concentration of ammonium formate is about 5 mM. In another aspect, the concentration of ammonium formate is between about 5 mM to about 10 mM.

In one aspect, the flow rate of the mobile phase for SEC is between about 0.125 mL/min and about 0.2 mL/min. In another aspect, the flow rate of the mobile phase for SEC is about 0.2 mL/min. In one aspect, the column temperature for SEC is between about 50° C. to about 70° C. In another aspect, the column temperature for SEC is about 60° C.

In one aspect, the SEC is coupled to the mass spectrometer. In one aspect, the mass spectrometer comprises a heated electrospray ionization source, an electrospray ionization source, a nano-electrospray ionization source, a microflow-nanospray electrospray ionization source, or a desorption electrospray ionization source. In another aspect, the mass spectrometer comprises a time-of-flight mass analyzer, a magnetic/electric sector mass analyzer, a quadrupole mass analyzer, or an orbitrap mass analyzer.

In one aspect, the method further comprises quantifying the distribution of heterogeneity of the protein components of the viral capsid based on the intensity of mass signals obtained by the mass spectrometry.

In one aspect, the separated protein components are detected by fluorescence and/or absorbance

In another exemplary embodiment, a method is provided for detecting or quantifying a nucleic acid in a viral vector, comprising: subjecting a sample comprising the viral vector to size exclusion chromatography under a denaturing mobile phase to separate the nucleic acid from protein components of a viral vector; and detecting the protein components by ultraviolet absorbance to detect and quantify the nucleic acid in the viral vector. In one aspect, the nucleic acid is DNA or RNA.

In one aspect, the ultraviolet absorbance is at a wavelength of 260 nm and 280 nm. In another aspect, the ultraviolet detector is connected in tandem with a fluorescence detector. In one aspect, the fluorescence is detected at an excitation wavelength of 280 nm, and an emission wavelength of 348 nm.

In one aspect, the viral vector is an adeno-associated virus (AAV) vector. In another aspect, the AAV vector comprises a serotype selected from a group consisting of AAV1, AAV2, AAV3, AAV4, AAV5, AAV6, AAV7, AAV8, AAV9, AAV10, AAV11, AAV12, variations thereof, and combinations thereof. In one aspect, the protein components of the viral capsid comprise VP1, VP2, and VP3 capsid proteins. In another aspect, the AAV vector is a recombinant AAV vector or an AAV vector encoding a heterologous transgene. In yet another aspect, the viral vector belongs to a viral family selected from the group consisting of Adenoviridae, Parvoviridae, Retroviridae, Baculoviridae, and Herpesviridae.

In one aspect, the denaturing mobile phase comprises acetonitrile, and formic acid in water. In one aspect, the concentration of acetonitrile is about 30%. In another aspect, the concentration of formic acid is about 1%.

In one aspect, the flow rate of the mobile phase for SEC is between about 0.125 mL/min and about 0.2 mL/min. In another aspect, the flow rate of the mobile phase for SEC is about 0.2 mL/min. In one aspect, the column temperature for SEC is between about 50° C. to about 70° C. In another aspect, the column temperature for SEC is about 60° C.

In one aspect, the SEC is coupled to the mass spectrometer. In one aspect, the mass spectrometer comprises a heated electrospray ionization source, an electrospray ionization source, a nano-electrospray ionization source, a microflow-nanospray electrospray ionization source, or a desorption electrospray ionization source. In another aspect, the mass spectrometer comprises a time-of-flight mass analyzer, a magnetic/electric sector mass analyzer, a quadrupole mass analyzer, or an orbitrap mass analyzer.

These, and other, aspects of the present disclosure will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the present disclosure.

The present disclosure provides a method for separating protein components of a viral capsid of a viral vector, comprising subjecting a sample comprising a viral vector to size exclusion chromatography (SEC) column under a denaturing mobile phase to separate the protein components of the viral capsid of the viral vector.

The present disclosure provides a method of identifying and/or characterizing at least one low molecular weight (LMW) antibody species in a sample, comprising subjecting the sample to size exclusion chromatography (SEC) column under a denaturing mobile phase to separate the LMW antibody species, and detecting the LMW antibody species by mass spectrometry to identify and/or characterize at least one LMW antibody species.

### BRIEF DESCRIPTION OF THE DRAWINGS

The summary, as well as the following detailed description, is further understood when read in conjunction with the appended drawings. For the purpose of illustrating the disclosed methods, there are shown in the drawing's exemplary embodiments of the methods; however, the methods are not limited to the specific embodiments disclosed. In the drawings:

FIG. 1A illustrates the MnESI configuration for denatured size exclusion chromatography (dSEC)-fluorescence (FLR)/ultraviolet (UV)-mass spectrometry (MS) detection of AAV serotypes.

FIG. 1B illustrates the heated electrospray (HESI) configuration for dSEC-FLR/UV-MS.

FIG. 2A shows dSEC-FLR separation using SEC columns of different pore size, 250 Å and 450 Å, according to an exemplary embodiment. The FLR traces were monitored using λₑₓ=280 nm and λₑₘ=348 nm, and the FLR peak identities were confirmed by accurate mass measurement and annotated.

FIG. 2B shows extracted ion chromatograms (XICs) of AAV8 VP1, VP2, and VP3 separated using an SEC column with a pore size of 450 Å, according to an exemplary embodiment.

FIG. 3 shows FLR chromatograms illustrating the effect of (a) %-ACN, (b) column temperature (° C.), and (c) ammonium formate (salt) concentration on dSEC separation of AAV8 VP(1-3), according to an exemplary embodiment.

FIG. 4 shows mass-to-charge (m/z) profiles illustrating the effect of (a) %-ACN, (b) column temperature (° C.), and (c) ammonium formate (salt) concentration on mass analysis of AAV8 VP(1-3).

FIG. 5A shows dSEC-FLR separation of AAV8 VP3 and AAV8 VP3 fragments corresponding to truncation between Asp659 and Pro660 with different column temperatures of 50, 60, and 70° C., according to an exemplary embodiment.

FIG. 5B shows XICs of AAV8 VP3 and AAV8 VP3 fragments corresponding to truncation between Asp659 and Pro660 at 70° C., according to an exemplary embodiment.

FIG. 6 shows dSEC-FLR chromatograms comparing the effect of SEC flow rate on the separation performance of AAV8 VP1(1-3) using an optimized, isocratic dSEC mobile phase consisting of 30% ACN, 1.0% FA, and 5 mM ammonium formate, and a column temperature of 60° C., according to an exemplary embodiment

FIG. 7 shows dSEC-FLR-MS analysis of AAV1, 2, 5, 6, 8, and 9 using an optimized, isocratic dSEC mobile phase consisting of 30% ACN, 1.0% FA, and 5 mM ammonium formate, according to an exemplary embodiment. FIG. 7A shows FLR chromatograms illustrating the separation of VP(1-3) across all the AAV serotypes tested, according to an exemplary embodiment. The insets show a 5x zoom-in on the y-axis to highlight the relative VP(1-3) intensity and separation performance between VP(1-3). FIG. 7B shows raw and corresponding deconvoluted MS spectra of AAV8 VP(1-3), with VP proteoforms annotated by accurate mass measurement, according to an exemplary embodiment. VP, viral protein; Ac, acetylation; p, monophosphorylation; pp, bisphosphorylation.

FIG. 8A shows representative dSEC-FLR, TIC-MS, and XICs of AAV1 VP1, VP2, and VP3, according to an exemplary embodiment.

FIG. 8B shows raw MS spectra of AAV1 VP1, VP2, and VP3, according to an exemplary embodiment.

FIG. 8C shows deconvoluted MS Spectra of AAV1 VP1, VP2, and VP3, according to an exemplary embodiment.

FIG. 9A shows representative dSEC-FLR, TIC-MS, and XICs of AAV2 VP1, VP2, and VP3, according to an exemplary embodiment.

FIG. 9B shows raw MS spectra of AAV2 VP1, VP2, and VP3, according to an exemplary embodiment.

FIG. 9C shows deconvoluted MS Spectra of AAV2 VP1, VP2, and VP3, according to an exemplary embodiment.

FIG. 10A shows representative dSEC-FLR, TIC-MS, and XICs of AAV5 VP1, VP2, and VP3, according to an exemplary embodiment.

FIG. 10B shows raw MS spectra of AAV5 VP1, VP2, and VP3, according to an exemplary embodiment.

FIG. 10C shows deconvoluted MS Spectra of AAV5 VP1, VP2, and VP3, according to an exemplary embodiment.

FIG. 11A shows representative dSEC-FLR, TIC-MS, and XICs of AAV6 VP1, VP2, and VP3, according to an exemplary embodiment.

FIG. 11B shows raw MS spectra of AAV6 VP1, VP2, and VP3, according to an exemplary embodiment.

FIG. 11C shows deconvoluted MS Spectra of AAV6 VP1, VP2, and VP3, according to an exemplary embodiment.

FIG. 12A shows representative dSEC-FLR, TIC-MS, and XICs of AAV8 VP1, VP2, and VP3, according to an exemplary embodiment.

FIG. 12B shows raw MS spectra of AAV8 VP1, VP2, and VP3, according to an exemplary embodiment.

FIG. 12C shows deconvoluted MS Spectra of AAV8 VP1, VP2, and VP3, according to an exemplary embodiment.

FIG. 13A shows representative dSEC-FLR, TIC-MS, and XICs of AAV9 VP1, VP2, and VP3, according to an exemplary embodiment.

FIG. 13B shows raw MS spectra of AAV9 VP1, VP2, and VP3, according to an exemplary embodiment.

FIG. 13C shows deconvoluted MS Spectra of AAV9 VP1, VP2, and VP3, according to an exemplary embodiment.

FIG. 14A shows comparison of AAV8 Process 1 and Process 2 lots analyzed by dSEC-FLR, according to an exemplary embodiment.

FIG. 14B shows comparison of AAV8 Process 1 and Process 2 lots analyzed by HILIC-FLR, according to an exemplary embodiment.

FIG. 14C shows total protein phosphorylation (Pₜₒₜ) calculated by mol Pi/mol protein for VP1 and VP2, determined by dSEC-MS and HILIC-MS, according to an exemplary embodiment.

FIG. 15 shows dSEC-UV traces at 260 nm of full AAV8 samples separated with varying concentration of ammonium formate (0-10 mM), according to an exemplary embodiment.

FIG. 16 shows comparison of AAV8 empty and full samples analyzed by dSEC-FLR/UV, according to an exemplary embodiment. UV detection was performed at both 260 and 280 nm.

FIG. 17A shows UV profiles of limited reduced mAb-1 separated on the 450 Å, 2.5 μm SEC column with 1% FA, 5 mM ammonium formate, 30% ACN mobile phase, according to an exemplary embodiment. MAb-1 was treated with 5 mM DTT for 30 seconds, 2 minutes and 10 minutes, followed with 15 mM IAM alkylation.

FIG. 17B shows deconvoluted mass spectra of H2L2, H2L, H2, HL, HC and LC with alkylation. CAM: carbamidomethyl.

FIG. 18A shows a comparison of dSEC-UV separation of an interchain reduced mAb using a Waters Xbridge BEH Premier SEC column, 250 Å, 1.7 μm, 4.6×300 mm, or a Waters GTxResolve Premier BEH SEC, 450 Å, 2.5 μm, 4.6×300 mm column, according to an exemplary embodiment. Comparison of the two columns using 0.1% TFA, 0.1% FA, and 30% ACN demonstrated that the 450 Å exhibits superior separation of the mAb LMW variants. Comparison of mobile phase compositions showed that 1.0% FA, 5 mM NH₄HCO₂ (ammonium formate), 30% ACN (mobile phase condition reported for dSEC analysis of AAVs), demonstrated that this condition has comparable separation to 0.1% TFA, 0.1% FA, and 30% ACN for mAbs, as illustrated by the similar UV profile and UV signal intensity.

FIG. 18B shows a comparison of dSEC-TIC profiles of the reduced mAb-1 using either 0.1% TFA, 0.1% FA, 30% ACN, or 1.0% FA, 5 mM NH₄HCO₂. 30% ACN, according to an exemplary embodiment. The latter demonstrates improved MS-ion intensity and MS-detection for larger LMW species such as H2L2, H2L, and H2.

FIG. 19A shows representative dSEC separation of IgG1 LMW variants, featuring 2 mAbs with K-LC (kappa light chains), and 2 mAbs with λ-LCs (lambda light chains). Zoom-in view (80×) of the UV profile highlights the robust separation of IgG1 dimer (P1), half mAb (P4), LC dimer (P5a/P5b), free LC (P6a/P6b), and VH domain fragment (P7).

FIG. 19B shows corresponding deconvoluted mass spectra of larger LMW variants, such as H2L, mAb missing Fab arm, and HC dimer (H2) from mAb-1 to mAb-4, showing a variety of post-translational modifications such as beta-elimination, cysteinylation, and glutathionylation. dSEC exhibited higher HC dimer in IgG1 mAbs containing lambda light chains.

FIG. 20A shows representative dSEC separation of IgG4 LMW variants, featuring 3 mAbs and 1 bispecific antibody (bsAb). Zoom-in view (200×) of the UV profile highlights the robust separation of IgG4 dimer (P1), half mAb (P4), Fab fragment (P5), free LC (P6), and VH/CH3 domain fragment (P7a/P7b).

FIG. 20B shows corresponding deconvoluted mass spectra of larger LMW variants, such as H2L, mAb missing Fab arm, mAb missing CH3 domain, and bsAb missing VH domain, showing a variety of post-translational modifications such as beta-elimination, cysteinylation, s-homocysteinylation, and glutathionylation.

FIG. 21 shows corresponding deconvoluted mass spectra of LMW variants and intact mAb, such as mAb Dimer (P1), intact mAb (P2), half mAb (P4), LC dimer and Fab (P5), free LC (P6), and VH domain (P7).

FIG. 22 shows corresponding deconvoluted mass spectra of LMW variants and intact mAb, such as mAb Dimer (P1), intact mAb (P2), half mAb (P4), Fab (P5), free LC (P6), and VH/CH3 domain (P7).

FIG. 23A shows dSEC-UV traces of an IgG1 mAb under thermal stress (40° C.) at t=0 and t=28 days separation. Zoom-in view (80×) of the UV profile shows the changes in LMW profile as a result of thermal stress.

FIG. 23B shows corresponding deconvoluted mass spectra of P3 and P5 at t=0 and t=28 days, showing the increase in mAb minus Fab arm, and Fab arm as a result of thermal stress. dSEC-MS can sensitively detect sequential truncation above the hinge region.

FIG. 24A shows dSEC-UV traces of an IgG4 mAb under thermal stress (40° C.) at t=0 and t=28 days separation. Zoom-in view (80×) of the UV profile shows the changes in LMW profile as a result of thermal stress.

FIG. 24B shows corresponding deconvoluted mass spectra of P3 at t=0 and t=28 days, showing the increase in IgG4 mAb minus CH3 domain.

FIG. 24C shows corresponding deconvoluted mass spectra of P7 at t=0 and t=28 days, showing the increase in IgG4 CH3 domain.

FIG. 24D shows corresponding deconvoluted mass spectra of P3 at t=0 and t=28 days, showing the increase in IgG4 mAb minus Fab.

FIG. 24E shows corresponding deconvoluted mass spectra of P5 at t=0 and t=28 days, showing the increase in IgG4 Fab.

FIG. 24F shows corresponding deconvoluted mass spectra of P6 at t=0 and t=28 days, showing the increase in truncated LC.

### DETAILED DESCRIPTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

Recombinant adeno-associated viruses (AAVs) are the leading platform for in vivo gene therapy with several FDA approved products to date, due to their low immunogenicity and effective transduction in a variety of cell types. The structure of the AAV capsid is composed of sixty copies of three different viral proteins (VPs) consisting of an approximately 1:1:10 (VP1:VP2:VP3) molar ratio, forming an icosahedral package which encapsulates a single-stranded DNA payload containing a gene of interest.

The stoichiometric ratio of VP(1-3) is a critical factor for maintaining AAV product integrity and significantly influences the ability for AAVs to deliver its genetic payload effectively. Specifically, low levels of VP1 and VP2 have been linked to reduced transduction efficacy, as the specific N-terminal sequences of VP1 and VP2 contain regions essential for nuclear internalization and genome release (Joshua C. Grieger et al., Journal of virology, 2006, 80(11), 5199-5210). Furthermore, capsid proteins can be intentionally altered through introduction of specific point mutations, undergo sequence truncations, or acquire post-translational modifications (PTMs), such as phosphorylation and deamidation, which all impact transduction efficiency. Therefore, intact mass spectrometry (MS)-based characterization of AAVs are invaluable for providing a comprehensive “bird's-eye view” of the VP proteoform landscape, enabling confirmation of AAV serotype identity, identification of unexpected VP proteoforms and their stoichiometry, and quantification of quality-associated PTMs.

Reversed-phase liquid chromatography (RPLC) and hydrophilic interaction liquid chromatography (HILIC) coupled to MS are the most implemented intact LC-MS techniques for AAV serotype confirmation and PTM characterization. To achieve optimal separation of VP(1-3), these chromatography modes have typically required the use of strong ion-pairing agents, such as difluoroacetic acid (DFA) or trifluoroacetic acid (TFA), to minimize unwanted analyte-stationary phase secondary interactions. However, compared to formic acid (FA), which is more volatile and typically favored in MS-based applications, these strong ion-pairing additives suppress MS signal intensity, making them suboptimal for highly sensitive MS analysis. Additionally, DFA (pKa: 1.33) and TFA (pKa: 0.52) are more acidic than FA (pKa: 3.75); therefore, these reagents commonly induce protein backbone hydrolysis at a higher rate under elevated column temperature conditions, which are commonly utilized for AAV VP(1-3) separation. As AAVs are typically formulated at relatively lower total protein concentrations (˜ng/μl range considering ˜1×10¹³ vector genomes/mL), LC-MS methods that can accommodate limited sample consumption requirements and allow sensitive detection of product quality attributes are highly desirable for AAV characterization.

Size exclusion chromatography (SEC) offers a compelling alternative to the adsorption-based chromatography modes such as RPLC and HILIC, for the separation of AAV capsid proteins based on their size or hydrodynamic volume. As a non-adsorption based chromatography mode, SEC prevails in method simplicity and wide applicability over other LC methods. In particular, SEC is highly compatible with a variety of solvent systems, is tolerable towards sample formulation buffers and is typically performed using isocratic conditions, which greatly simplifies chromatographic method development. Thus, SEC methods can be modulated to be suitable towards a variety of denatured protein applications coupled to online MS detection. While SEC is commonly used in the biopharmaceutical industry for the analysis of protein-based therapeutics, SEC-MS analysis of intact AAV VP components has failed to gain any traction to date, presumably due to the difficulty in achieving adequate resolution of VP(1-3) monomers.

The present disclosure provides methods and systems for characterizing protein components of a viral capsid of a viral vector. In particular, the methods comprise subjecting a viral vector to size exclusion chromatography under a denaturing mobile phase to separate the protein components. Protein components can be detected by fluorescence to determine the relative abundance of the protein components separated by SEC. Further, the protein components can be subjected to mass spectrometry to determine the masses of the protein components, which can be compared to theoretical masses to determine heterogeneity of a viral vector. Further, the present disclosure also provides methods for detecting or quantifying a nucleic acid in a viral vector. In particular, the methods comprise subjecting a viral vector to size exclusion chromatography under a denaturing mobile phase to separate the nucleic acid from protein components of the viral vector, and detecting the components by ultraviolet absorbance to detect and quantify the nucleic acid in the viral vector.

The present disclosure developed an effective and broadly applicable SEC-based method for intact AAV capsid protein analysis under denaturing conditions. In particular, this method features a novel mobile phase selection and simultaneous online detection using fluorescence (FLR), ultraviolet (UV 260/280 nm) absorption, and MS. Through the optimization of chromatography parameters which affect the SEC separation behavior of VP components, this method can effectively separate VP(1-3) from a variety of AAV serotypes without prior sample denaturation or sample clean-up steps. Denatured SEC (dSEC) can also be used for facile quantitation of VP stoichiometry while maintaining the ability to accurately monitor truncated species and PTMs, such as phosphorylation, due to the sensitive detection enabled by FLR and MS. Additionally, with slight adjustment to the mobile phase, the dSEC method can be used to separate and detect the DNA payload from an AAV sample and enable lot-to-lot comparisons in relative abundance of DNA content.

Also disclosed herein are effective and broadly applicable SEC-based methods for detection of low molecular weight (LMW) mAb species. In particular, these methods feature a novel mobile phase selection and simultaneous online detection using fluorescence (FLR), ultraviolet (UV 260/280 nm) absorption, and MS. Through the optimization of chromatography parameters which affect the SEC separation behavior, these methods can effectively detect LMW species from both IgG1, IgG4, and bispecific antibody molecules.

Without wishing to be bound by theory, dSEC provides high sensitivity of LMW mAb species, and provides simplified grouping of the LMW species into specific classes for facilitated identification.

Without wishing to be bound by theory, dSEC can be effectively applied to both IgG1 (kappa and lambda molecules) and IgG4 and bispecific antibodies and can also be used to analyze molecules subjected to thermal stress conditions. These methods include observable LMW species pathway formations that are common in IgG1 and IgG4 molecules.

The present disclosure provides methods of identifying and/or characterizing at least one low molecular weight (LMW) antibody species in a sample. The methods comprise subjecting the sample to size exclusion chromatography (SEC) column under a denaturing mobile phase to separate the LMW antibody species. The methods further comprise detecting the LMW antibody species by mass spectrometry to identify and/or characterize at least one LMW antibody species.

The denaturing mobile phase may comprise acetonitrile, formic acid, and ammonium formate in water.

The denaturing mobile phase may comprise acetonitrile at a concentration of about 30%. The denaturing mobile phase may comprise acetonitrile at a concentration of about 10%. The denaturing mobile phase may comprise acetonitrile at a concentration of about 20%. The denaturing mobile phase may comprise acetonitrile at a concentration of about 25%. The denaturing mobile phase may comprise acetonitrile at a concentration of about 35%. The denaturing mobile phase may comprise acetonitrile at a concentration of about 40%.

The denaturing mobile phase may comprise formic acid at a concentration of about 1%. The denaturing mobile phase may comprise formic acid at a concentration of about 0.1%. The denaturing mobile phase may comprise formic acid at a concentration of about 0.5%. The denaturing mobile phase may comprise formic acid at a concentration of about 0.7%. The denaturing mobile phase may comprise formic acid at a concentration of about 1.2%. The denaturing mobile phase may comprise formic acid at a concentration of about 1.5%. The denaturing mobile phase may comprise formic acid at a concentration of about 2%. The denaturing mobile phase may comprise formic acid at a concentration of about 3%. The denaturing mobile phase may comprise formic acid at a concentration of about 4%. The denaturing mobile phase may comprise formic acid at a concentration of about 5%.

The denaturing mobile phase may comprise ammonium formate at a concentration of between about 5 mM to about 10 mM. The denaturing mobile phase may comprise ammonium formate at a concentration of about 1 mM. The denaturing mobile phase may comprise ammonium formate at a concentration of about 2 mM. The denaturing mobile phase may comprise ammonium formate at a concentration of about 3 mM. The denaturing mobile phase may comprise ammonium formate at a concentration of about 4 mM. The denaturing mobile phase may comprise ammonium formate at a concentration of about 5 mM. The denaturing mobile phase may comprise ammonium formate at a concentration of about 6 mM. The denaturing mobile phase may comprise ammonium formate at a concentration of about 7 mM. The denaturing mobile phase may comprise ammonium formate at a concentration of about 8 mM. The denaturing mobile phase may comprise ammonium formate at a concentration of about 9 mM. The denaturing mobile phase may comprise ammonium formate at a concentration of about 10 mM. The denaturing mobile phase may comprise ammonium formate at a concentration of about 11 mM. The denaturing mobile phase may comprise ammonium formate at a concentration of about 12 mM. The denaturing mobile phase may comprise ammonium formate at a concentration of about 15 mM. The denaturing mobile phase may comprise ammonium formate at a concentration of about 20 mM.

The flow rate of the mobile phase for the SEC may be between about 0.125 mL/min and about 0.2 mL/min. The flow rate of the mobile phase for the SEC may be about 0.05 mL/min. The flow rate of the mobile phase for the SEC may be about 0.1 ml/min. The flow rate of the mobile phase for the SEC may be about 0.125 mL/min. The flow rate of the mobile phase for the SEC may be about 0.135 mL/min. The flow rate of the mobile phase for the SEC may be about 0.145 mL/min. The flow rate of the mobile phase for the SEC may be about 0.155 mL/min. The flow rate of the mobile phase for the SEC may be about 0.165 mL/min. The flow rate of the mobile phase for the SEC may be about 0.175 mL/min. The flow rate of the mobile phase for the SEC may be about 0.185 mL/min. The flow rate of the mobile phase for the SEC may be about 0.195 mL/min. The flow rate of the mobile phase for the SEC may be about 0.2 mL/min. The flow rate of the mobile phase for the SEC may be about 0.225 mL/min. The flow rate of the mobile phase for the SEC may be about 0.250 mL/min. The flow rate of the mobile phase for the SEC may be about 0.275 ml/min. The flow rate of the mobile phase for the SEC may be about 0.300 mL/min.

The SEC may be coupled to a mass spectrometer. The mass spectrometer may comprise a heated electrospray ionization source, an electrospray ionization source, a nano-electrospray ionization source, a microflow-nanospray electrospray ionization source, or a desorption electrospray ionization source. The mass spectrometer may comprise a time-of-flight mass analyzer, a magnetic/electric sector mass analyzer, a quadrupole mass analyzer, or an orbitrap mass analyzer.

Unless described otherwise, all technical and scientific terms used herein have the same meaning as commonly understood by one of ordinary skill in the art to which this disclosure belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule.

In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., 28 Biotechnology And Genetic Engineering Reviews 147-176 (2012), the entirety of which is herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FRI, CDR1, FR2, CDR2, FR3, CDR3, and FR4. In different embodiments of the present disclosure, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′) 2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” (bsAbs) includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or ka-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (e.g., bispecific antibodies, bsAbs), antibodies with additional specificities such as trispecific antibody and KIH Trispecific are also contemplated.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

As used herein, the term “sample” refers to a mixture of molecules that comprises at least one viral vector, such as an AAV vector, that is subjected to manipulation in accordance with the methods of the disclosure, including, for example, separating, analyzing, extracting, concentrating, profiling and the like.

As used herein, the terms “viral vector” or “vector” refer to a viral (e.g., AAV) particle that can be used to mediate delivery of a nucleic acid to a host cell, either in vitro or in vivo. A viral vector is composed of at least one viral capsid protein, and may include a viral genome (e.g., viral DNA) that is packaged within the viral capsid. The term “capsid” or “capsid protein” refers to the protein shell of a virus, which can enclose genetic material. Alternatively, in some contexts, the term “vector” may be used to refer to only the viral genome or to a recombinant plasmid. The term “viral genome” refers to a nucleic acid sequence that is flanked by cis acting nucleic acid sequences that mediate the packaging of the nucleic acid into a viral capsid. For AAVs, for example, it is known that inverted terminal repeats” (ITRs) located at the 5′ and 3′ end of the viral genome have this function and that the ITRs can mediate the packaging of heterologous, for example, non-wild type virus genomes, into a viral capsid. Such a viral genome can be replicated and packaged into infectious viral particles when present in a host cell that has been infected with a suitable helper virus (or that is expressing suitable helper functions) and that is expressing AAV rep and cap gene products (e.g. AAV Rep and Cap proteins).

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA).

Oligonucleotides may be modified, e.g., comprise a modified nucleotide, a modified internucleoside linkage, and/or a modified sugar moiety, or combinations thereof. In some embodiments, particular nucleotide modification(s) may be incorporated that render an oligonucleotide more resistant to nuclease digestion than the native oligoribonucleotide or oligodeoxynucleotide molecules; such modified polynucleotides survive intact for a longer time than unmodified polynucleotides. Exemplary modified polynucleotides include those comprising modified backbones, for example, modified internucleoside linkages such as, methyl phosphonates, phosphotriesters, phosphorothioates short chain alkyl or cycloalkyl intersugar linkages heterocyclic intersugar linkages or short chain heteroatomic or. As such, the oligonucleotide may be stabilized against nucleolytic degradation, e.g., via incorporation of a modification, e.g., a nucleotide modification.

As used herein, the term “AAV” is an abbreviation for adeno-associated virus, and may be used to refer to the virus itself or derivatives thereof. The term covers all subtypes and both naturally occurring and recombinant forms, except where required otherwise. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8). Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, for example, interferon-mediated responses; (iii) wild type AAVs have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected).

As used herein, the term “AAV vector” or “AAV viral particle” refers to a viral particle composed of at least one AAV capsid protein (typically by all of the capsid proteins of a wild-type AAV). The AAV capsid protein is made up of three capsid protein monomers, VP1, VP2, and VP3. Sixty copies of these three VP proteins interact in a 1:1:10 ratio to form the viral capsid. VP1 covers the whole of VP2 protein in addition to a ˜137 amino acid N-terminal region (VPlu), VP2 covers the whole of VP3 in addition to ˜65 amino acid N-terminal region (VP½ common region). The three capsid proteins share a conserved amino acid sequence of VP3, which in some cases is the region beginning at amino acid position 217 (e.g., AA 217-736).

An AAV vector may include a rAAV vector. The term “recombinant AAV (rAAV)” refers to an AAV genome in which part or all of the AAV rep and cap genes have been replaced with one or more heterologous sequences (e.g. nucleic acid sequence not of AAV origin).

The term “heterologous” means derived from a genotypically distinct entity from that of the rest of the entity to which it is compared or into which it is introduced or incorporated. For example, a nucleic acid introduced by genetic engineering techniques into a different cell type is a heterologous nucleic acid (and, when expressed, can encode a heterologous polypeptide). Similarly, a cellular sequence (e.g., a gene or portion thereof) that is incorporated into a viral vector is a heterologous nucleotide sequence with respect to the vector.

The AAV “rep” gene refers to nucleic acid sequences that encode the non-structural proteins required for replication and production of virus. The AAV “cap” gene refer to nucleic acid sequences that encode capsid proteins that form, or contribute to the formation of, the capsid, or protein shell of the virus. In the case of AAV, the capsid protein may be VP1, VP2, or VP3.

While AAV is described in this disclosure as a model virus or viral particle, it is contemplated that the disclosed methods can be applied to profile a variety of viruses, e.g., the viral families, subfamilies, and genera. In some aspects, the viral capsid, virus, or viral particle belongs to a viral family selected from the group consisting of Adenoviridae, Parvoviridae, Retroviridae, Baculoviridae, and Herpesviridae. In some aspects, the viral capsid, virus, or viral particle belongs to a viral genus selected from the group consisting of Atadenovirus, Aviadenovirus, Ichtadenovirus, Mastadenovirus, Siadenovirus, Ambidensovirus, Brevidensovirus, Hepandensovirus, Iteradensovirus, Penstyldensovirus, Amdoparvovirus, Aveparvovirus, Bocaparvovirus, Copiparvovirus, Dependoparvovirus, Erythroparvovirus, Protoparvovirus, Tetraparvovirus, Alpharetrovirus, Betaretrovirus, Deltaretrovirus, Epsilonretrovirus, Gammaretrovirus, Lentivirus, Spumavirus, Alphabaculovirus, Betabaculovirus, Deltabaculovirus, Gammabaculovirus, Iltovirus, Mardivirus, Simplexvirus, Varicellovirus, Cytomegalovirus, Muromegalovirus, Proboscivirus, Roscolovirus, Lymphocryptovirus, Macavirus, Percavirus, and Rhadinovirus.

In some exemplary embodiments, the present application includes the use of liquid chromatography techniques, such as size exclusion chromatography.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase liquid chromatography, ion-exchange chromatography, size exclusion chromatography, affinity chromatography, hydrophobic interaction chromatography, hydrophilic interaction chromatography, or mixed-mode chromatography. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

In some exemplary embodiments, SEC can be performed using a denaturing mobile phase. As used herein, the term “denaturing mobile phase” refers to a mobile phase that can disrupt non-covalent interactions in an analyte.

In some exemplary embodiment, the mobile phase used in size exclusion chromatography can comprise an organic solvent. In some specific embodiments, the organic solvent is acetonitrile. In some aspect, the mobile phase comprises a concentration of acetonitrile from about 20% to about 40%, or about 20%, about 25%, about 30%, about 35%, or about 40%.

In some exemplary embodiments, the mobile phase used in size exclusion chromatography can comprise an acid. In some specific embodiments, the mobile phase can comprise formic acid, difluoroacetic acid, dichloroacetic acid, trichloroacetic acid, and methanesulfonic acid. In some specific embodiments, the mobile phase comprises a concentration of formic acid from about 0.1% to about 1.0%, or about 0.1%, about 0.2% about 0.3%, about 0.4% about 0.5%, about 0.6%, about 0.7%, about 0.8%, about 0.9%, or about 1.0%.

In some exemplary embodiments, the mobile phase used to obtain the eluate from size exclusion chromatography can comprise a volatile salt. In some specific embodiments, the mobile phase can comprise ammonium acetate, ammonium bicarbonate, or ammonium formate, or combinations thereof. In some aspects, the mobile phase comprises a concentration of ammonium formate from about 5 mM to about 10 mM, or about 5 mM, about 5.5 mM, about 6 mM, about 6.5 mM, about 7 mM, about 7.5 mM, about 8 mM, about 8.5 mM, about 9 mM, about 9.5 mM, or about 10 mM.

In some exemplary embodiments, an elevated column temperature is used in size exclusion chromatography. In some aspects, the column temperature is from about 50° C. to about 70° C., or about 50° C., about 55° C., about 60° C., about 65° C., or about 70° C.

In some exemplary embodiments, a flow rate from about 0.125 mL/min to about 0.2 mL/min is used in size exclusion chromatography. In some aspects, the flow rate is about 0.125 mL/min, about 0.130 mL/min, about 0.140 mL/min, about 0.150 mL/min, about 0.160 mL/min, about 0.170 mL/min, about 0.180 mL/min, about 0.190 mL/min, or about 0.200 mL/min.

As used herein, the term “mass spectrometry” includes the use of a device capable of identifying specific molecular species and measuring their accurate masses. The term is meant to include any molecular detector into which a polypeptide or peptide may be characterized. The mass spectrometer can be coupled to a liquid chromatography system, wherein the mass spectrometer is capable of performing LC-MS (liquid chromatography-mass spectrometry). A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application. Non-limiting examples of ion sources include electrospray ionization (ESI), atmospheric pressure ionization (API), matrix assisted laser desorption ionization (MALDI), laser desorption ionization (LDI), and desorption electrospray ionization (DESI). The term “mass analyzer” refers to a device that can separate species, that is, atoms, molecules, or clusters, according to their mass. Non-limiting examples of mass analyzers include time-of-flight (TOF), magnetic/electric sector, quadrupole mass filter (Q), quadrupole ion trap (QIT), orbitrap, Fourier transform ion cyclotron resonance (FTICR), and also the technique of accelerator mass spectrometry (AMS).

It is understood that the present disclosure is not limited to any of the aforesaid protein(s), recombinant protein(s), antibody(ies), sample(s), vector(s), nucleic acid(s), AAV(s), AAV vector(s), capsid protein(s), chromatographic method(s), or mass spectrometer(s), and any protein(s), recombinant protein(s), antibody(ies), sample(s), vector(s), nucleic acid(s), AAV(s), AAV vector(s), capsid protein(s), chromatographic method(s), or mass spectrometer(s) can be selected by any suitable means.

The present disclosure will be more fully understood by reference to the following examples. They should not, however, be construed as limiting the scope of the disclosure.

### EMBODIMENTS

The following list of embodiments is intended to complement, rather than displace or supersede, the previous descriptions.

### Embodiments Set 1

1. A method for separating protein components of a viral capsid of a viral vector, comprising:
(a) obtaining a sample comprising said viral vector; and
(b) subjecting said sample to size exclusion chromatography under a denaturing mobile phase to separate said protein components of said viral capsid of said viral vector.
   2. The method of embodiment 1, wherein said viral vector is an adeno-associated virus (AAV) vector.
   3. The method of embodiment 2, wherein said AAV vector comprises a serotype selected from a group consisting of AAV1, AAV2, AAV3, AAV4, AAV5, AAV6, AAV7, AAV8, AAV9, AAV10, AAV11, AAV12, variants thereof, and combinations thereof. 4. The method of embodiment 2, wherein said protein components of said viral capsid comprise VP1, VP2, and VP3 capsid proteins.
   5. The method of embodiment 2, wherein said AAV vector is a recombinant AAV vector or an AAV vector encoding a heterologous transgene.
   6. The method of embodiment 1, wherein said viral vector belongs to a viral family selected from the group consisting of Adenoviridae, Parvoviridae, Retroviridae, Baculoviridae, and Herpesviridae.
   7. The method of embodiment 1, wherein said denaturing mobile phase comprises acetonitrile, formic acid, and ammonium formate in water.
   8. The method of embodiment 7, wherein a concentration of said acetonitrile is about 30%.
   9. The method of embodiment 7, wherein a concentration of said formic acid is about 1%.
   10. The method of embodiment 7, wherein a concentration of ammonium formate is about 5 mM.
   11. The method of embodiment 7, wherein a concentration of ammonium formate is between about 5 mM to about 10 mM.
   12. The method of embodiment 1, wherein a flow rate of said mobile phase for said SEC is between about 0.125 mL/min and about 0.2 mL/min.
   13. The method of embodiment 1, wherein a flow rate of said mobile phase for said SEC is about 0.2 mL/min.
   14. The method of embodiment 1, wherein a column temperature for said SEC is between about 50° C. to about 70° C.
   15. The method of embodiment 1, wherein a column temperature for said SEC is about 60° C.
   16. The method of embodiment 1, wherein said fluorescence is detected using an excitation wavelength of 280 nm, and an emission wavelength of 348 nm.
   17. A method for determining stoichiometry of protein components of a viral capsid of a viral vector, comprising:
(a) subjecting a sample comprising said viral vector to size exclusion chromatography under a denaturing mobile phase to separate said protein components; and
(b) detecting said protein components by fluorescence to determine the relative abundance of said protein components separated by SEC, thereby determining the stoichiometry of said protein components of said viral capsid of said viral vector.
   18. The method of embodiment 17, wherein said viral vector is an adeno-associated virus (AAV) vector.
   19. The method of embodiment 18, wherein said AAV vector comprises a serotype selected from a group consisting of AAV1, AAV2, AAV3, AAV4, AAV5, AAV6, AAV7, AAV8, AAV9, AAV10, AAV11, AAV12, variations thereof, and combinations thereof.
   20. The method of embodiment 18, wherein said protein components of said viral capsid comprise VP1, VP2, and VP3 capsid proteins.
   21. The method of embodiment 18, wherein said AAV vector is a recombinant AAV vector or an AAV vector encoding a heterologous transgene.
   22. The method of embodiment 17, wherein said viral vector belongs to a viral family selected from the group consisting of Adenoviridae, Parvoviridae, Retroviridae, Baculoviridae, and Herpesviridae.
   23. The method of embodiment 17, wherein said denaturing mobile phase comprises acetonitrile, formic acid, and ammonium formate in water.
   24. The method of embodiment 23, wherein a concentration of said acetonitrile is about 30%, 25. The method of embodiment 23, wherein a concentration of said formic acid is about 1%.
   26. The method of embodiment 23, wherein a concentration of ammonium formate is about 5 mM.
   27. The method of embodiment 23, wherein a concentration of ammonium formate is about 5 mM to about 10 mM.
   28. The method of embodiment 17, wherein a flow rate of a mobile phase for said SEC is between about 0.125 mL/min and about 0.2 mL/min.
   29. The method of embodiment 17, wherein a flow rate of a mobile phase for said SEC is about 0.2 mL/min.
   30. The method of embodiment 17, wherein a column temperature for said SEC is between about 50° C. to about 70° C.
   31. The method of embodiment 17, wherein a column temperature for said SEC is about 60° C.
   32. The method of embodiment 17, further comprising subjecting said proteins components to mass spectrometry to identify said protein components.
   33. The method of embodiment 32, wherein said SEC is coupled to said mass spectrometer.
   34. The method of embodiment 32, wherein said mass spectrometer comprises a heated electrospray ionization source, an electrospray ionization source, a nano-electrospray ionization source, a microflow-nanospray electrospray ionization source, or a desorption electrospray ionization source.
   35. The method of embodiment 32, wherein said mass spectrometer comprises a time-of-flight mass analyzer, a magnetic/electric sector mass analyzer, a quadrupole mass analyzer, or an orbitrap mass analyzer.
   36. The method of embodiment 32, further comprising detecting said protein components by UV absorbance to determine the relative abundance of said protein components, wherein said UV detector is connected in tandem with said fluorescence detector.
   37. A method for determining heterogeneity of protein components of a viral capsid of a viral vector, comprising:
(c) subjecting a sample comprising said viral vector to size exclusion chromatography under a denaturing mobile phase to separate said protein components; and
(d) subjecting said proteins components to mass spectrometry to determine the masses of said protein components, wherein said determined masses of said protein components are compared to theoretical masses to determine heterogeneity.
   38. The method of embodiment 37, wherein said heterogeneity comprises one or more of mixed serotypes, variant capsids, capsid protein amino acid substitutions, truncated capsid proteins, or modified capsid proteins.
   39. The method of embodiment 37, wherein said viral vector is an adeno-associated virus (AAV) vector.
   40. The method of embodiment 39, wherein said AAV vector comprises a serotype selected from a group consisting of AAV1, AAV2, AAV3, AAV4, AAV5, AAV6, AAV7, AAV8, AAV9, AAV10, AAV11, AAV12, variations thereof, and combinations thereof.
   41. The method of embodiment 39, wherein said protein components of said viral capsid comprise VP1, VP2, and VP3 capsid proteins.
   42. The method of embodiment 39, wherein said AAV particle is a recombinant AAV vector or an AAV vector encoding a heterologous transgene.
   43. The method of embodiment 37, wherein said viral vector belongs to a viral family selected from the group consisting of Adenoviridae, Parvoviridae, Retroviridae, Baculoviridae, and Herpesviridae.
   44. The method of embodiment 37, wherein said denaturing mobile phase comprises acetonitrile, formic acid, and ammonium formate in water.
   45. The method of embodiment 44, wherein a concentration of said acetonitrile is about 30%.
   46. The method of embodiment 44, wherein a concentration of said formic acid is about 1%.
   47. The method of embodiment 44, wherein a concentration of ammonium formate is about 5 mM.
   48. The method of embodiment 44, wherein a concentration of ammonium formate is between about 5 mM to about 10 mM.
   49. The method of embodiment 37, wherein a flow rate of a mobile phase for said SEC is between about 0.125 mL/min and about 0.2 mL/min.
   50. The method of embodiment 37, wherein a flow rate of a mobile phase for said SEC is about 0.2 mL/min.
   51. The method of embodiment 37, wherein a column temperature for said SEC is between about 50° C. to about 70° C.
   52. The method of embodiment 37, wherein a column temperature for said SEC is about 60° C.
   53. The method of embodiment 37, wherein said SEC is coupled to said mass spectrometer.
   54. The method of embodiment 37, wherein said mass spectrometer comprises a heated electrospray ionization source, an electrospray ionization source, a nano-electrospray ionization source, a microflow-nanospray electrospray ionization source, or a desorption electrospray ionization source.
   55. The method of embodiment 37, wherein said mass spectrometer comprises a time-of-flight mass analyzer, a magnetic/electric sector mass analyzer, a quadrupole mass analyzer, or an orbitrap mass analyzer.
   56. The method of embodiment 37, further comprising quantifying the distribution of heterogeneity of said protein components of said viral capsid based on the intensity of mass signals obtained by said mass spectrometry.
   57. The method of embodiment 37, wherein said separated protein components are detected by fluorescence and/or absorbance.
   58. A method for detecting or quantifying a nucleic acid in a viral vector, comprising:
(a) subjecting a sample comprising said viral vector to size exclusion chromatography under a denaturing mobile phase to separate the nucleic acid from protein components of a viral vector; and
(b) detecting said components by ultraviolet absorbance to detect and quantify said nucleic acid in said viral vector.
   59. The method of embodiment 58, wherein said nucleic acid is DNA or RNA.
   60. The method of embodiment 58, wherein said ultraviolet absorbance is detected at a wavelength of 260 nm and 280 nm.
   61. The method of embodiment 58, wherein said ultraviolet detector is connected in tandem with a fluorescence detector.
   62. The method of embodiment 61, wherein said fluorescence is detected at an excitation wavelength of 280 nm, and an emission wavelength of 348 nm. 63. The method of embodiment 58, wherein said viral vector is an adeno-associated virus (AAV) vector.
   64. The method of embodiment 63, wherein said AAV vector comprises a serotype selected from a group consisting of AAV1, AAV2, AAV3, AAV4, AAV5, AAV6, AAV7, AAV8, AAV9, AAV10, AAV11, AAV12, variations thereof, and combinations thereof.
   65. The method of embodiment 63, wherein said protein components of said viral capsid comprise VP1, VP2, and VP3 capsid proteins.
   66. The method of embodiment 63, wherein said AAV particle is a recombinant AAV vector or an AAV vector encoding a heterologous transgene.
   67. The method of embodiment 58, wherein said viral vector belongs to a viral family selected from the group consisting of Adenoviridae, Parvoviridae, Retroviridae, Baculoviridae, and Herpesviridae.
   68. The method of embodiment 58, wherein said denaturing mobile phase comprises acetonitrile, and formic acid in water.
   69. The method of embodiment 68, wherein a concentration of said acetonitrile is about 30%.
   70. The method of embodiment 68, wherein a concentration of said formic acid is about 1%.
   71. The method of embodiment 58, wherein a flow rate of a mobile phase for said SEC is between about 0.125 mL/min and about 0.2 mL/min.
   72. The method of embodiment 58, wherein a flow rate of a mobile phase for said SEC is about 0.2 mL/min.
   73. The method of embodiment 58, wherein a column temperature for said SEC is between about 50° C. to about 70° C.
   74. The method of embodiment 58, wherein a column temperature for said SEC is about 60° C.
   75. The method of embodiment 58, wherein said SEC is coupled to said mass spectrometer.
   76. The method of embodiment 58, wherein said mass spectrometer comprises a heated electrospray ionization source, an electrospray ionization source, a nano-electrospray ionization source, a microflow-nanospray electrospray ionization source, or a desorption electrospray ionization source.
   77. The method of embodiment 58, wherein said mass spectrometer comprises a time-of-flight mass analyzer, a magnetic/electric sector mass analyzer, a quadrupole mass analyzer, or an orbitrap mass analyzer.

### Embodiments Set 2

1. A method for separating protein components of a viral capsid of a viral vector, comprising:
subjecting a sample comprising a viral vector to size exclusion chromatography (SEC) column under a denaturing mobile phase to separate the protein components of the viral capsid of the viral vector.
   2. The method of embodiment 1, wherein the viral vector is an adeno-associated virus (AAV) vector comprising a serotype selected from AAV1, AAV2, AAV3, AAV4, AAV5, AAV6, AAV7, AAV8, AAV9, AAV10, AAV11, AAV12, variants thereof, and combinations thereof.
   3. The method of embodiment 1, wherein the protein components of the viral capsid comprise VP1, VP2, and VP3 capsid proteins.
   4. The method of embodiment 3, further comprising determining the stoichiometry of the VP1, VP2, and VP3 capsid proteins by detecting the capsid proteins by fluorescence to determine the relative abundance of the capsid proteins, thereby determining the stoichiometry of the VP1, VP2, and VP3 capsid proteins.
   5. The method of embodiment 1, wherein the denaturing mobile phase comprises acetonitrile, formic acid, and ammonium formate in water.
   6. The method of embodiment 5, wherein the concentration of acetonitrile is about 30%.
   7. The method of embodiment 5, wherein the concentration of formic acid is about 1%.
   8. The method of embodiment 5, wherein the concentration of ammonium formate is between about 5 mM to about 10 mM.
   9. The method of embodiment 1, wherein a flow rate of the mobile phase for the SEC is between about 0.125 mL/min and about 0.2 mL/min.
   10. The method of embodiment 1, further comprising subjecting the protein components to mass spectrometry to identify the separated protein components.
   11. A method of identifying and/or characterizing at least one low molecular weight (LMW) antibody species in a sample, comprising:
(a) subjecting the sample to size exclusion chromatography (SEC) column under a denaturing mobile phase to separate the LMW antibody species; and
(b) detecting the LMW antibody species by mass spectrometry to identify and/or characterize at least one LMW antibody species.
   12. The method of embodiment 11, wherein the denaturing mobile phase comprises acetonitrile, formic acid, and ammonium formate in water.
   13. The method of embodiment 12, wherein the concentration of acetonitrile is about 30%.
   14. The method of embodiment 12, wherein the concentration of formic acid is about 1%.
   15. The method of embodiment 12, wherein the concentration of ammonium formate is between about 5 mM to about 10 mM.
   16. The method of embodiment 11, wherein a flow rate of the mobile phase for the SEC is between about 0.125 mL/min and about 0.2 mL/min.
   17. The method of embodiment 11, wherein the SEC is coupled to a mass spectrometer.
   18. The method of embodiment 17, wherein the mass spectrometer comprises a heated electrospray ionization source, an electrospray ionization source, a nano-electrospray ionization source, a microflow-nanospray electrospray ionization source, or a desorption electrospray ionization source.
   19. The method of embodiment 17, wherein the mass spectrometer comprises a time-of-flight mass analyzer, a magnetic/electric sector mass analyzer, a quadrupole mass analyzer, or an orbitrap mass analyzer.

### EXAMPLES

Materials and reagents. AAV2-Empty, AAV5-CMV-Luciferase, AAV6-CMV-Luciferase were obtained from Virovek (Houston, TX, USA). AAV1-Full, AAV8 (Empty and Full), AAV9-Full were generated by triple transfection of HEK 293 Ts at Regeneron Pharmaceuticals, Inc. (Tarrytown, NY, USA). Samples were dispersed into small aliquots (20 μl) to avoid freeze-thaw cycles, stored at −80° C., and analyzed within 24 hours of thawing.

Optima LC-MS grade acetonitrile (ACN) was sourced from Fisher Scientific (Dublin, Ireland). Deionized water (Milli-Q water) was obtained from a Milli-Q integral water purification system and was sourced by Millipore Sigma (Burlington, MA, USA). LC-MS grade ammonium formate was obtained from Millipore Sigma (St. Louis, MO, USA). LC-MS grade formic acid was obtained from Honeywell Fluka (Morris Plains, NJ, USA).

Analytical Instrumentation. Intact AAV capsids were analyzed using a Vanquish Horizon ultrahigh pressure liquid chromatography (UHPLC) instrument consisting of a Binary Pump (VH-P10-A), Sample Manager (Dual Split Sampler HT (VH-A40A), column compartment (VH-C10-A) with Active Preheater (6732.0110), Fluorescence Detector F (VF-D51-A) equipped with a Micro bio flowcell (6079.4330), and UV-VIS detector (VF-D40-A) equipped with a semi-micro bio flow cell (6077.0300) and a deuterium lamp (6077.1111). All LC lines were Thermo Scientific™ Viper™ Capillary MP35N.

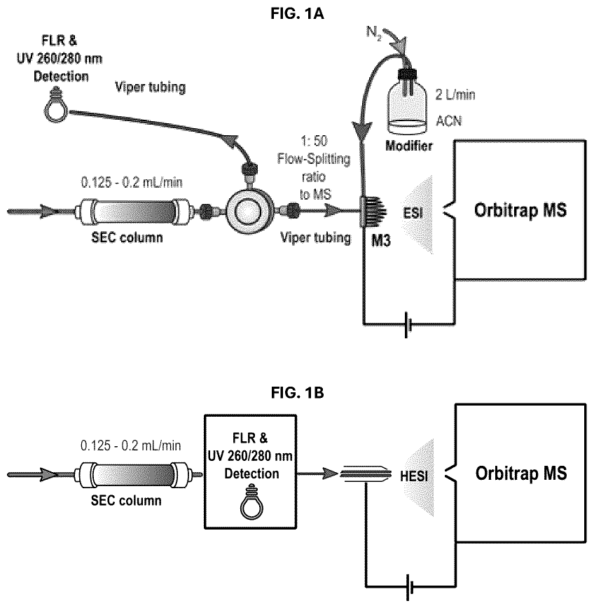

The Vanquish UHPLC was coupled to a Thermo Scientific™ Orbitrap Exploris 480 Mass Spectrometer (MS) interfaced with either an adjustable heated electrospray ionization (HESI) probe or a Microflow-Nanospray Electrospray Ionization (MnESI) source and an 8-nozzle, 10 μm ID Microfabricated Monolithic Multinozzle (M3) emitter (Newomics, Berkeley, CA) for MS analysis. The MnESI source was equipped with acetonitrile as the dopant gas, and Newomics 1:50 flow splitter (#FSK-01). Schemes of the interfaces that accommodate the HESI source or MnESI source are depicted in FIGS. 1A and 1B. Details on the nanoESI platform using the MnESI source can also be found in Yuctian Yan, Tao Xing, Shunhai Wang, and Ning Li. Versatile, sensitive, and robust native LC-MS platform for intact mass analysis of protein drugs. J. Am. Soc. Mass Spectrom. 2020, 31(10), 2171-2179, which is incorporated by reference in its entirety.

Denaturing size exclusion chromatography (dSEC). AAV samples were analyzed using a GTxResolve Premier BEH SEC column, 4.6×300 mm, 2.5 μm, 450 Å (Waters Corporation, Milford, MA, USA). 1-5 μL of each AAV serotype (˜2E13-1E14 cp/mL) were injected neat as intact capsids and denatured on column. After optimization of the mobile phase composition, an isocratic gradient of 30% acetonitrile, 1.0% formic acid, and 5 mM ammonium formate at 0.125 ml/min (˜35 min LC runs) was used for VP(1-3) separation for all AAV serotypes. For optimal DNA detection, an isocratic gradient of 30% acetonitrile, 1.0% formic acid was used at a flow rate of 0.200 ml/min corresponding to ˜20 min LC runs. New columns were initially conditioned with 20 column volumes of mobile phase at 60° C. before consistent use.

UHPLC instrument module settings were used as follows: autosampler temperature set to 5° C.; column compartment and active solvent preheater temperature set to 60° C.; fluorescence detector excitation wavelength (λex) set to 280 nm, emission wavelength (λem) set to 348 nm, acquisition rate of 10.0 Hz, lamp set to “long-life mode”, flow cell temperature set to 30° C., and detector sensitivity set to 1. The UV absorbance wavelengths measured were 260 nm and 280 nm at an acquisition rate of 2.0 Hz.

Global MS parameters used on the Orbitrap Exploris 480 instrument were as follows: Intact Protein was selected for the application mode, low pressure was selected for pressure mode, and liquid chromatography was selected for the infusion mode.

The MnESI ion source properties used are as follows: full scan MS1 was run in positive ESI ion mode with a static spray voltage of 3800V. Sheath gas was set to 10, with auxiliary and sweep gas set to 0. The ion transfer tube temperature was 320° C. The scan range was 750-4000 m/z. Samples were analyzed with an Orbitrap resolution of 7,500 (at m/z 200) corresponding to a 16-ms transient signal. The RF lens was set at 60%, the normalized ACG target (%) was set at 300, the maximum injection time (ms) was 200, with 10 microscans. Data were collected in profile mode. In-source CID was set to 15V.

The HESI ion source properties used are as follows: full scan MS1 was run in positive ESI ion mode with a static spray voltage of 3500V. Sheath gas was set to 10, and auxiliary gas set to 7. The vaporizer temperature was 275° C. and the ion transfer tube temperature was 320° C. The scan range was 750-4000 m/z. Samples were analyzed with an Orbitrap resolution of 7,500 (at m/z 200) corresponding to a 16-ms transient signal. The RF lens was set at 60%, the normalized ACG target (%) was set at 300, the maximum injection time (ms) was 200, with 10 microscans recorded. Data were collected in profile mode. In-source CID was set to 15V.

Hydrophilic interaction liquid chromatography-fluorescence (HILIC-FLR). AAV capsid separation using HILIC-FLR-MS was performed following the LC-MS parameters described by Anita P. Liu, Shailin K. Patel, Tao Xing, Yuetian Yan, Shunhai Wang, and Ning Li. Characterization of Adeno-Associated Virus Capsid Proteins Using Hydrophilic Interaction Chromatography Coupled with Mass Spectrometry. J Pharm Biomed Anal. 2020, 189, 113481, with column compartment and active solvent preheater set to 50° C.

Data analysis. Intact mass spectra from dSEC-MS analysis were deconvoluted using Intact Mass software from Protein Metrics (Cupertino, CA).

### Example 1. Optimization of dSEC-FLR-MS for Separating AAV Capsid Proteins

SEC separates biomolecules based on their hydrodynamic radii by allowing the biomolecules to diffuse through a stationary phase consisting of spherical, porous particles with precisely controlled pore sizes. In a complex mixture, the largest analytes have limited access to the pores of the stationary phase and therefore elute first. They are followed by smaller analytes, which typically elute in order of decreasing size. Due to the relative simplicity of intact AAV capsids, which consist of three viral proteins with distinct molecular-weight (MW) differences (VP1, approximately 82 kDa; VP2, approximately 66 kDa; VP3, approximately 59 kDa), we investigated whether a SEC-based method could predictably and distinctly separate the viral protein components to facilitate their MS-characterization and analysis.

To enable size-based separation of individual viral proteins rather than intact AAV capsid assemblies, we employed a denaturing mobile phase condition in the analysis of reduced and non-reduced monoclonal antibodies, but modified to counteract TFA-induced ion suppression (Hongcheng Liu, Georgeen Gaza-Bulseco, and Chris Chumsae, Journal of the American Society for Mass Spectrometry, 2009, 20(12), 2258-2264). This condition consisted of 1% formic acid (FA) in 30% acetonitrile (ACN) and 69% water, with the column temperature elevated to 60° C. to aid in the dissociation of AAV capsids on-column. Formic acid (FA) is used in MS-based applications due to its volatility and its role in imparting charge during electrospray ionization, thereby boosting MS sensitivity towards analyte detection. As FA is both a relatively weaker acid and ion-pairing reagent, it was applied at a higher concentration to replace and compensate for the strong ion-pairing effect from TFA. Under this denaturing condition, we assessed individual SEC columns that shared similar dimensions (4.6×300 mm, 2.5 μm), but differed in pore size (250 Å and 450 Å). Our column evaluation utilized protein standards such as NISTmAb (approximately 145 kDa) and bovine serum albumin (BSA, approximately 66 kDa), alongside an intact AAVS sample. Both FLR detection, which uses the intrinsic fluorescence of tryptophan to detect proteins, and MS detection were used to assess SEC column performance and confirm FLR peak assignments, respectively.

SEC-MS analysis of intact AAV VP components has failed to gain any traction to date, possibly due to the difficulty in achieving adequate resolution of VP(1-3) monomers. These studies describe an effective and broadly applicable SEC-based method for intact AAV capsid protein analysis under denaturing conditions with novel mobile phase selection and simultaneous online detection using fluorescence (FLR), ultraviolet (UV 260/280 nm) absorption and MS. The study demonstrates that this method is capable of separating VP(1-3) from a variety of AAV serotypes, without prior sample denaturation or sample clean-up steps. Denatured SEC (dSEC) can be used for facile quantitation of VP stoichiometry while maintaining the ability to accurately monitor truncated species and PTMs such as phosphorylation, due to the sensitive detection enabled by FLR and MS. Additionally, the dSEC method can be used to separate and detect the DNA payload from an AAV sample and enable lot-to-lot comparisons in relative abundance of DNA content.

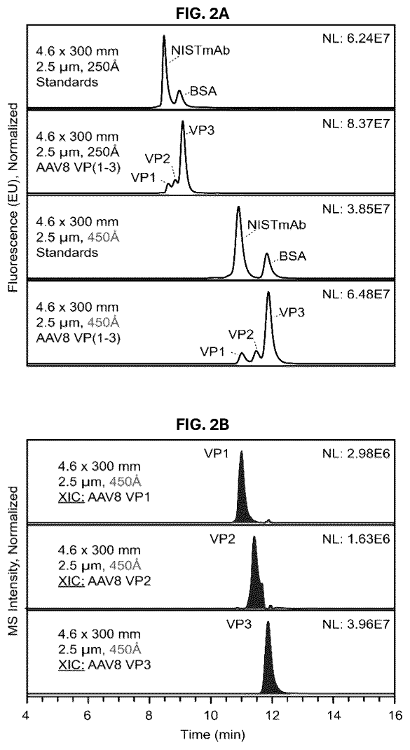

The SEC column featuring a 450 Å pore size demonstrated superior separation capabilities compared to the 250 Å column under the denaturing conditions. This was demonstrated by the improved peak-to-peak separation between the NISTmAb and BSA protein standards, and a reduction in co-elution among the AAV8 VP(1-3) components (FIG. 2A). While the AAV8 VP3 exhibited a similar retention time to the BSA standard, the dissociated VPI exhibited a similar retention time to the NISTmAb standard despite the stark difference in molecular weight. Due to their varying stability profiles, this observation likely suggests differences in the extent to which these proteins unfold under the selected denaturing condition.

Additionally, as proteins vary in shape (e.g., globular, rod-like or flexible chains), their hydrodynamic radii and their resulting SEC-elution profile may not correlate exactly with molecular weight trends. However, the longer elution times observed using the 450 Å column suggests greater pore-accessibility for VP(1-3), resulting in improved protein separation over the 250 Å column under denaturing conditions, which is consistent with a size-exclusion based chromatography mechanism.

Additionally, the extracted ion chromatograms (XICs) of the individual AAV8 VP components showed a SEC elution profile that aligned with the descending order of viral protein molecular weight, with VPI eluting first, followed by VP2, and then VP3 (FIG. 2B). Despite the relatively small molecular weight differences among the VP(1-3) components, their distinct separation by dSEC held great promise and warranted further method investigation.

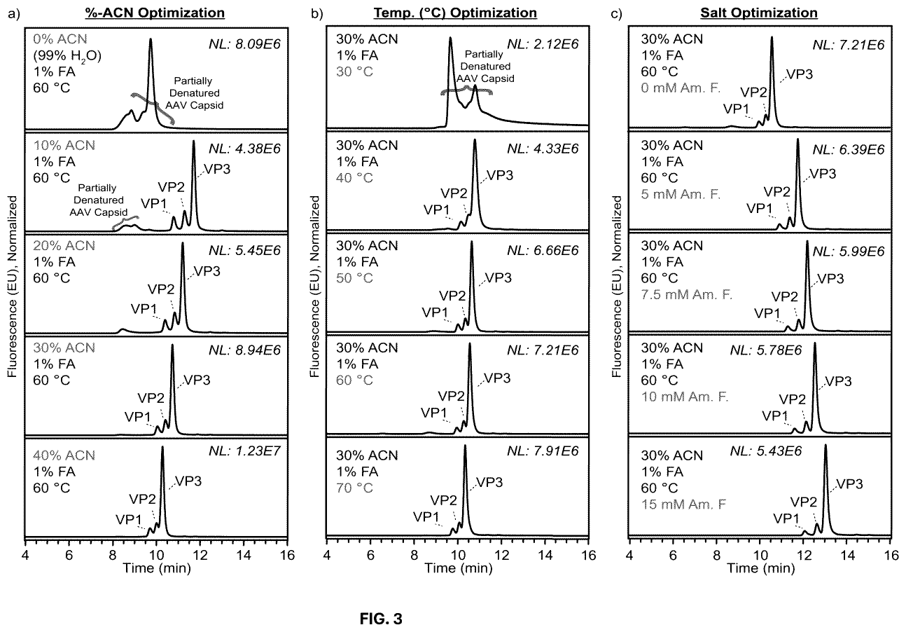

Following the initial success in denaturing intact AAV capsids and separating their individual VP components using SEC under denaturing conditions, the effect of various mobile phase additives and chromatography parameters on the separation performance of AAV8 VP components were further investigated (FIG. 3). The selection of mobile phase additives was guided by their potential to impact secondary interactions in SEC, such as hydrophobic and electrostatic interactions. To evaluate the effect of organic solvents in the mobile phase on VP separation, we systematically varied the composition of ACN in the mobile phase. Interestingly, despite the increase in column temperature to 60° C., the use of 1% FA in water (0% ACN, pH 2.2) alone appeared insufficient to fully denature AAV8 on-column and produced a broad elution profile of partially denatured capsids. Incrementally increasing the ACN concentration from 0 to 40% significantly reduced the amount of partially or incompletely denatured AAV8 capsids, and enabled the resolution and MS detection of individual VP(1-3) components (FIG. 3, see a). The enhancement in peak sharpness with higher ACN concentrations suggests a reduction in the hydrophobic interactions between VP components and the stationary phase. However, as ACN composition increased, both earlier elution times and increased co-elution between the different VPs were observed, likely due to increased protein unfolding in presence of higher organic modifier content.

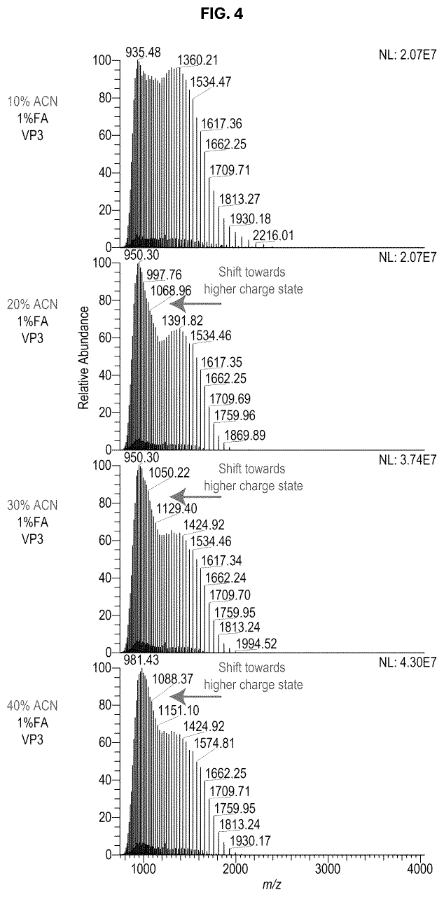

Additionally, the measured m/z profiles of the VPs exhibited a shift towards higher charge states with increase in ACN concentration, further supporting the notion that enhanced protein denaturation and unfolding contributes to decreased SEC resolution (FIG. 4). A mobile phase of 30% ACN was ultimately selected as the starting point for further optimization because it significantly reduced the presence of partially denatured AAV8 capsids, and achieved satisfactory resolution of the VP(1-3) species.

The effect of column temperature on VP separation was also evaluated since elevated temperatures have been demonstrated to induce AAV capsid denaturation. Using a mobile phase of 30% ACN and 1% FA, the column temperature was varied from 30° C. to 70° C. As seen in FIG. 3 (see b), a column temperature of 30° C. appeared insufficient to fully denature intact AAV8 capsids, resulting in the detection of some dissociated VPs and partially denatured AAV8 capsids. At 40° C., the appearance of partially denatured AAV8 capsids substantially decreased, but poor resolution between VP2 and VP3 was observed. As temperature increased further, distinct resolution and overall earlier elution was observed for the VP components, suggesting that higher temperatures induced further protein unfolding and conformational changes.

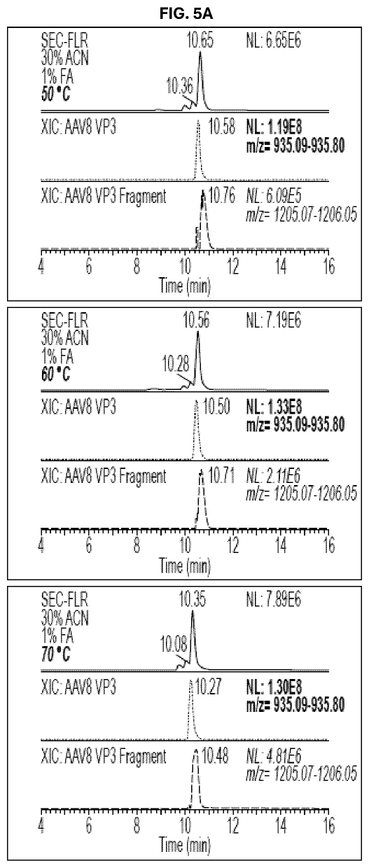

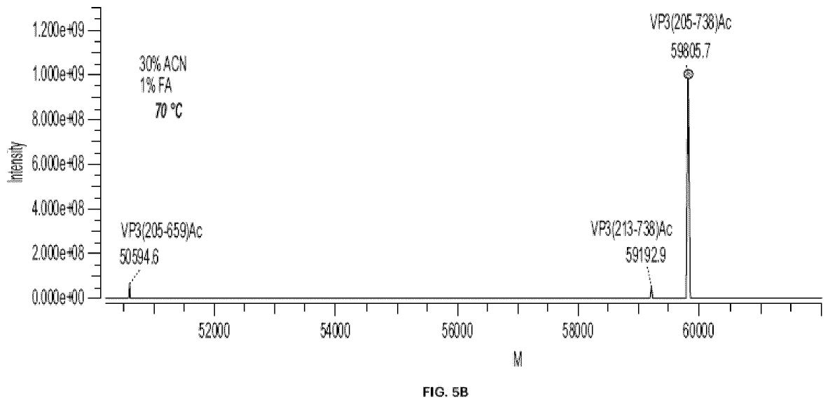

Additionally, an increase in the presence of a C-terminal VP3 fragment corresponding to truncation between Asp659 and Pro660 was observed with higher column temperatures. Using MS-based quantitation, the fragment was estimated to be 1.7% at 60° C., and 4.5% at 70° C. (FIGS. 5A and 5B, Table 1). This D|P clipping product is a well-known assay artifact frequently observed under low pH and elevated temperature conditions, and can be effectively mitigated by adjusting the column temperature and the mobile phase composition (Josef Vlasak, Roxana Ionescu, Fragmentation of monoclonal antibodies, mAbs, 2011, 3(3), 253-63). The detection of this artifact illustrates the importance of selecting an optimal, elevated temperature to aid in AAV capsid dissociation and to enhance the resolution between different VP components while avoiding analytical artifacts such as D|P fragments.

TABLE 1
Summary of MS quantitation of AAV8 VP3 and
AAV8 VP3 fragment at different temperature.

Relative Abundance
(%-MS intensity)

| Column Temp. | AAV 8 VP3 | AAV8 VP3 |
|---|---|---|
| (° C.) | (205-738) Ac | (205-659) Ac |
| 50° C. | 99.50% | 0.50% |
| 60° C. | 98.30% | 1.70% |
| 70° C. | 95.50% | 4.50% |

Although less common, salt additives in the mobile phase of dSEC can significantly enhance protein separation and chromatography resolution. Specifically, the increased ionic strength in the mobile phase can influence the hydrodynamic radii of proteins in their denatured state by mitigating the electrostatic repulsion forces along the protein chain, thereby altering their elution profiles. Ammonium salts, such as ammonium formate and ammonium acetate, are popular mobile phase additives, due to their desirable ion-pairing capability, volatility, and MS compatibility.

To investigate the advantages of a salt additive, mobile phases consisting of 30% ACN, 1% FA, varying concentrations of ammonium formate ranging from 0 to 15 mM were evaluated. As the concentration of ammonium formate increased, we observed a concomitant increase in retention time and improvement in peak-to-peak separation across the different VP monomers (FIG. 3, see c). This observation supports the notion that without the “ion pairing” effect of salt, denatured VPs adopt a more linear conformation and larger effective hydrodynamic radii, resulting in relatively earlier elution. As more salt was introduced into the mobile phase, electrostatic repulsion along the VP monomers was partially mitigated, which led to decreased effective hydrodynamic radii, enhanced pore permeability and increased SEC-elution volume. Only minimal resolution improvement was gained between VP1-3 components when the ammonium formate concentration was increased from 5 mM to 15 mM. Considering ammonium additives at higher concentrations (≥10 mM) may cause significant MS-ion suppression and compromise the detection sensitivity, a final concentration of ammonium formate was selected at 5 mM.

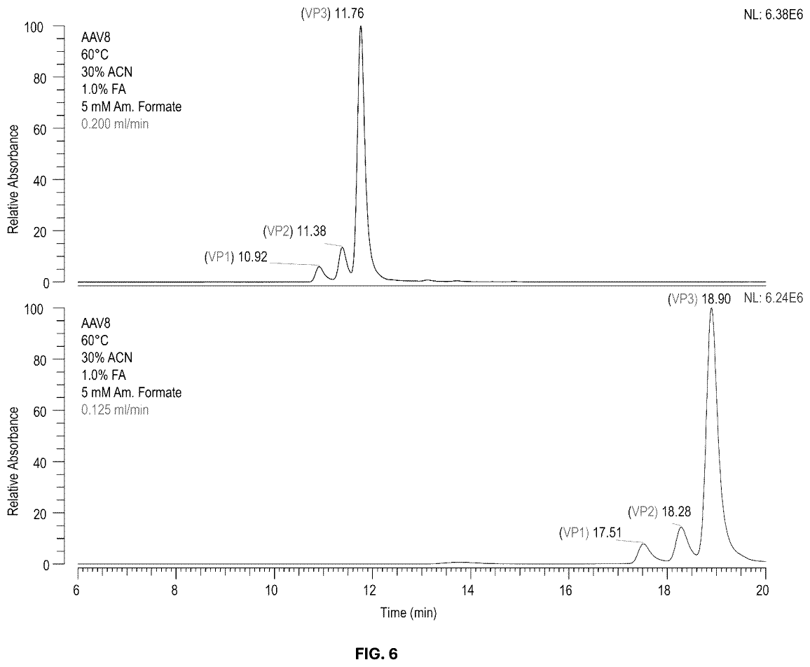

It was hypothesized that SEC resolution between the different VP components can be further improved by decreasing the flow rate. As shown in FIG. 6, decreasing the flow rate allowed for higher peak to peak resolution among the VP monomers at the tradeoff of slightly longer analysis times and broader elution peaks, due to slower diffusion of the VPs within the pores of the SEC column.

### Example 2. Applicability of dSEC-FLR-MS Among Common AAV Serotypes

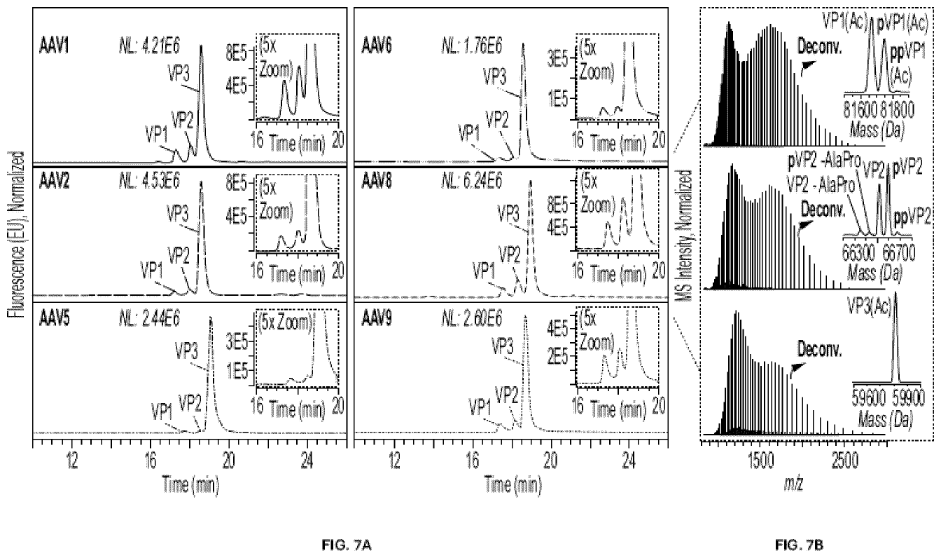

AAV exists in multiple serotypes that vary in both biophysical and biological activities, a number of which (AAV 1, 2, 5, 6, 8, and 9) are commonly selected to accommodate specific therapeutic requirements. Therefore, the broad applicability of an analytical method in characterizing different AAV serotypes is an important criterion to consider during method development. To investigate whether this optimized dSEC-MS method would apply to different AAV serotypes, a variety of commonly used AAV serotypes (AAV 1, 2, 5, 6, 9) along with the previously evaluated AAV8, were subjected to online dSEC-FLR-MS analysis (FIG. 7, see a).

Notably, using the same SEC column and mobile phase/chromatography parameters, consistent and effective VP(1-3) separations were achieved for all tested AAV serotypes (FIG. 7, see a). This result highlights the broad applicability of the size-based separation of intact AAV VP components under denaturing conditions, despite serotype-dependent differences in physicochemical properties, such as hydrophobicity. Moreover, due to the distinct elution of VP1, VP2, and VP3 species, their ratio can be estimated using the corresponding FLR intensities to reflect the stoichiometry of each AAV sample analyzed (Table 2).

TABLE 2
FLR-based quantitation of VP stoichiometry present in
the AAV1, 2, 5, 6, 8 and 9 samples analyzed by dSEC.

| | | | | Normalized | Avg. Capsid |
|---|---|---|---|---|---|
| | VP1 | VP2 | VP3 | VP(1-3) | VP(1-3) |
| Serotype | Rel. Abundance (% Peak Area) | | | Ratio | Ratio |
| AAV1 | 8.6% | 11.4% | 80.0% | 1.0:1.3:9.3 | 5.2:6.8:48.0 |
| AAV2 | 3.9% | 5.5% | 90.6% | 1.0:1.4:23.5 | 2.3:3.3:54.4 |
| AAV5 | 1.9% | 2.2% | 95.9% | 1.0:1.1:49.3 | 1.2:1.3:57.5 |
| AAV6 | 2.7% | 2.8% | 94.5% | 1.0:1.0:35.2 | 1.6:1.6:56.7 |
| AAV8 | 7.1% | 12.1% | 80.8% | 1.0:1.7:11.4 | 4.2:7.3:48.5 |
| AAV9 | 7.7% | 7.9% | 84.4% | 1.0:1.0:11.0 | 4.6:4.7:50.7 |

Interestingly, most AAV serotype samples exhibited ratios close to the expected value of 1:1:10 (VP1: VP2: VP3), while others exhibited significantly lower VP1 and VP2 abundances. It is understood that different production methods or processes can lead to different expression levels of the three VPs, and that incorporation of VPs is a stochastic process, which can lead to different ratios of VPs. However, due to potential limitations arising from peak tailing and potential analyte recovery issues, the dSEC peak area-based quantitation may exhibit deviations from the true capsid stoichiometry. Nevertheless, the dSEC-FLR-MS method can be reliably used to provide quick batch-to-batch comparative assessments of AAV productions.

In addition to the effective VP(1-3) separation across different AAV serotypes, sensitive MS detection using the developed dSEC-MS method was also demonstrated with the successful detection of all VPs and their less abundant proteoforms. As exemplified using AAV8, strong MS signal and high-quality mass spectra of VP1, VP2, and VP3 were obtained with 200 ng (2E13 cp/mL×1 μL) of sample loading, allowing for the unbiased identification of both major and minor proteoforms in each peak (FIG. 7, see b, and Table 3). Specifically, VP3 was identified to primarily exist in the N-terminally acetylated form (VP3(Ac)), following excision of its initiator methionine and N-terminal acetylation of the following alanine. On the other hand, VP1 and VP2 were found to consist of two major proteoforms, including N-terminally acetylated VP1 without (VP1(Ac)) or with phosphorylation (pVP1(Ac)), and VP2 without (VP2) or with phosphorylation (pVP2), respectively.

TABLE 3
Comparison of the experimental and theoretical masses of capsid viral
proteins and their variants identified from dSEC-FLR-MS analysis
of AAV1, AAV2, AAV5, AAV6, AAV8, and AAV9 serotype samples.

| | AAV Capsid | | AAV | AAV | |
|---|---|---|---|---|---|
| AAV | Variant | | Theoretical | Observed | \|ΔMass\| |
| Serotype | Identity | | Mass (Da) | Mass (Da) | (Da) |
| AAV1 | VP1 | VP1 (2-736)Ac | 81286.1 | 81286.1 | 0.0 |
| | VP1 + | VP1 (2-736)Ac | 81366.1 | 81368.0 | 1.9 |
| | Phosphorylation | | | | |
| | N-term | VP2 (141-736) | 65925.3 | 65926.1 | 0.8 |
| | Fragment VP2 | | | | |
| | VP2 | VP2 (139-736) | 66093.5 | 66092.6 | 0.9 |
| | VP2 + | VP2 (139-736) | 66173.5 | 66173.9 | 0.4 |
| | Phosphorylation | | | | |
| | N-Term | VP3 (212-736)Ac | 58888.6 | 58887.9 | 0.7 |
| | Fragment VP3 | | | | |
| | VP3 | VP3 (204-736)Ac | 59517.3 | 59516.3 | 1.0 |
| AAV2 | VP1 | VP1 (2-735)Ac | 81555.5 | 81856.1 | 0.6 |
| | VP1 + | VP1 (2-735)Ac | 81935.5 | 81934.8 | 0.7 |
| | Phosphorylation | | | | |
| | VP2 | VP2 (139-735) | 66488.6 | 66488.6 | 0.0 |
| | VP2 + | VP2 (139-735) | 66568.6 | 66567.8 | 0.8 |
| | Phosphorylation | | | | |
| | VP2 * | VP2 + 171.6 Da | 66660.2 | — | — |
| | VP2 * | VP2 + 954.0 Da | 67442.6 | — | — |
| | C-Term | VP3 (206-735)Ac | 59802.3 | 59799.5 | 2.8 |
| | Fragment VP3 | | | | |
| | C-Term | VP3 (212-735)Ac | 59301.7 | 59300.9 | 0.8 |
| | Fragment VP3 | | | | |
| | VP3 | VP3 (204-735)Ac | 59974.5 | 59973.2 | 1.3 |
| AAV5 | VP1 | VP1(2-724)Ac | 80335.7 | 80333.6 | 2.1 |
| | VP2 | VP2(138-724) | 65283.4 | 65282.0 | 1.4 |
| | VP3 | VP3(194-724)Ac | 59463.1 | 59460.2 | 2.9 |
| AAV6 | VP1 | VP1 (2-736)Ac | 81322.2 | 81322.4 | 0.2 |
| | VP1 + | VP1 (2-736)Ac | 81402.2 | 81402.8 | 0.6 |
| | Phosphorylation | | | | |
| | VP2 | VP2 (139-736) | 66095.6 | 66094.7 | 0.9 |
| | VP2 + | VP2 (139-736) | 66175.6 | 66175.1 | 0.5 |
| | Phosphorylation | | | | |
| | VP2 * | VP2 + 171.4 Da | 66267.0 | — | — |
| | VP2 * | VP2 + 715.5 Da | 66811.1 | — | — |
| | VP3 | VP3 (204-736)Ac | 59519.4 | 59517.8 | 1.6 |
| | VP3 + | VP3 (204-736)Ac | 59599.4 | 59600.9 | 1.5 |
| | Phosphorylation | | | | |
| AAV8 | VP1 | VP1 (2-738)Ac | 81667.3 | 81668.3 | 1.0 |
| | VP1 + | VP1 (2-738)Ac + | 81747.3 | 81747.8 | 0.5 |
| | Phosphorylation | Phos | | | |
| | VP1 + | VP1 (2-738)Ac + | 81827.3 | 81829.1 | 1.8 |
| | Phosphorylation (2) | Phos(2) | | | |
| | N-term | VP2 (141-738) | 66350.6 | 66351.8 | 1.2 |
| | Fragment VP2 | | | | |
| | N-term | VP2 (141-738) | 66430.6 | 66431.6 | 1.0 |
| | Fragment VP2 + | | | | |
| | Phosphorylation | | | | |
| | VP2 | VP2 (139-738) | 66518.8 | 66518.3 | 0.5 |
| | VP2 + | VP2 (139-738) + | 66598.7 | 66597.8 | 0.9 |
| | Phosphorylation | Phos | | | |
| | VP2 + | VP2 (139-738) + | 66678.7 | 66681.8 | 3.1 |
| | Phosphorylation (2) | Phos (2) | | | |
| | N-term | VP3(213-738)Ac | 59192.7 | 59192.0 | 0.7 |
| | Fragment VP3 | | | | |
| | C-term | VP3(205-659)Ac | 50594.4 | 50594.0 | 0.0 |
| | Fragment VP3 | | | | |
| | VP3 | VP3 (205-738)Ac | 59805.4 | 59804.3 | 1.1 |
| AAV9 | VP1 | VP1 (2-736)Ac | 81289.9 | 81291.5 | 1.6 |
| | VP1 + | VP1 (2-736)Ac + | 81369.9 | 81373.1 | 3.2 |
| | Phosphorylation | Phos | | | |
| | VP2 | VP2 (139-736) | 66209.4 | 66209.3 | 0.1 |
| | VP2 + | VP2 (139-736) + | 66289.4 | 66226.1 | 2.7 |
| | Phosphorylation | Phos | | | |
| | VP3 | VP3 (204-736)Ac | 59732.3 | 59731.7 | 0.6 |
| | C-term | VP3 (204-657)Ac | 50469.2 | 50469.5 | 0.3 |
| | Fragment VP3 | | | | |

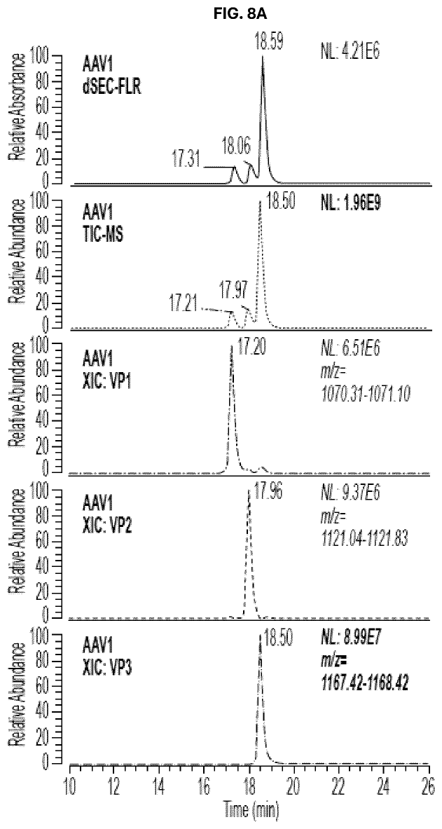

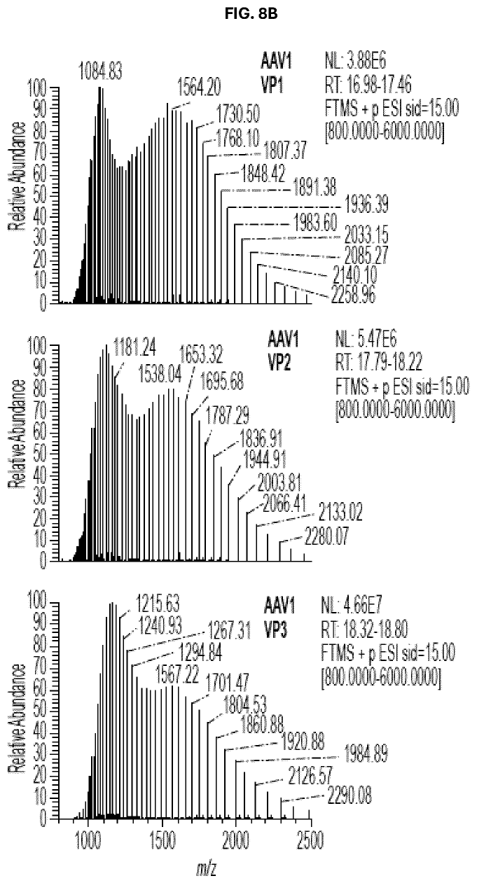

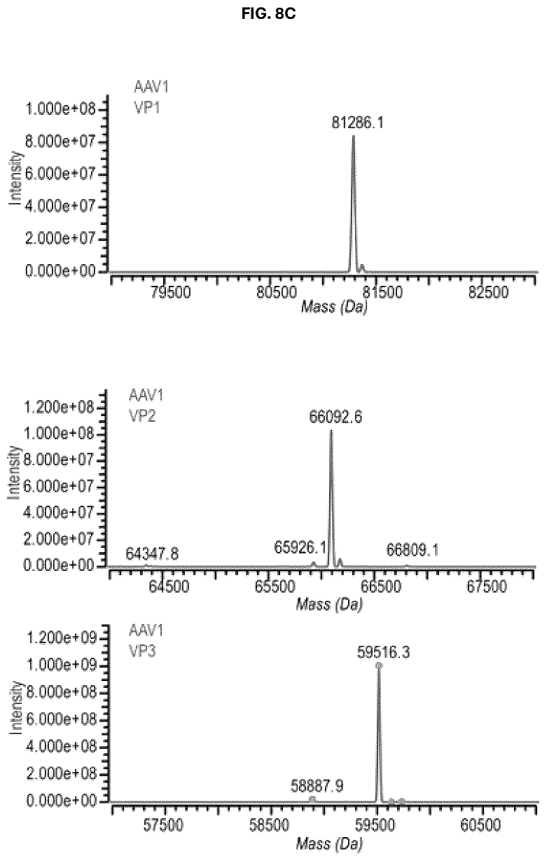

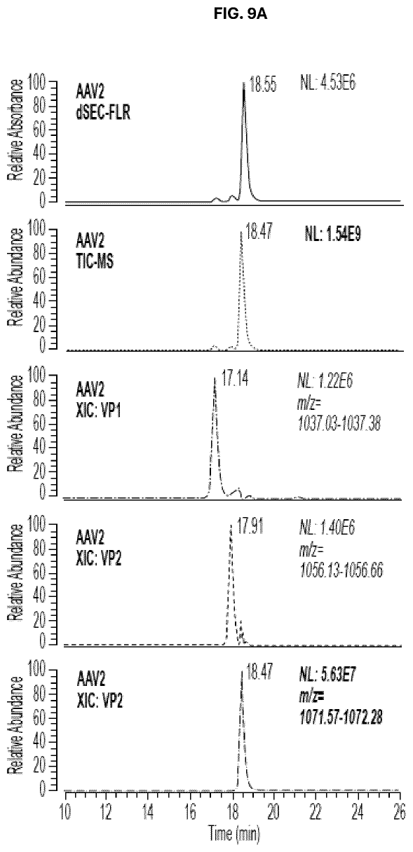

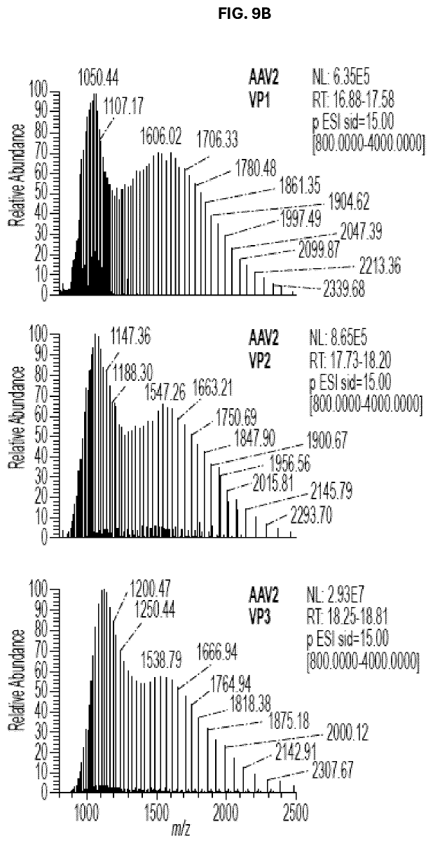

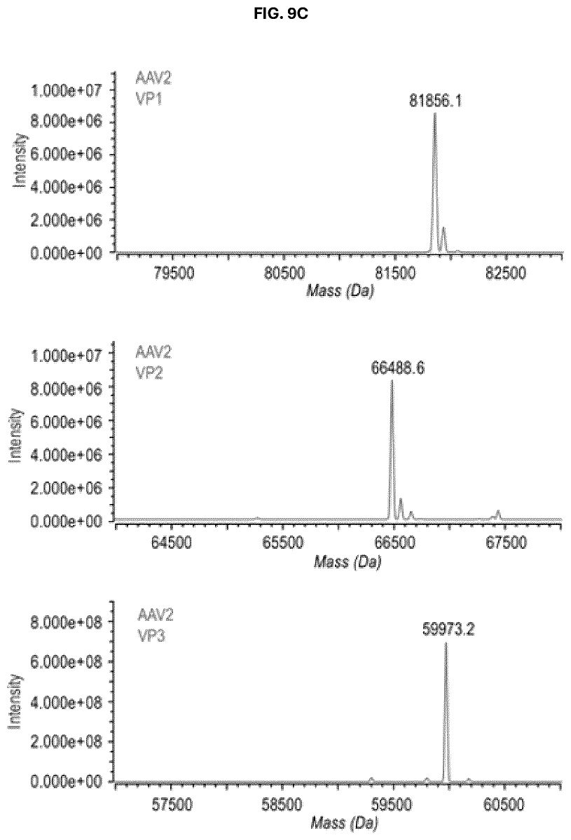

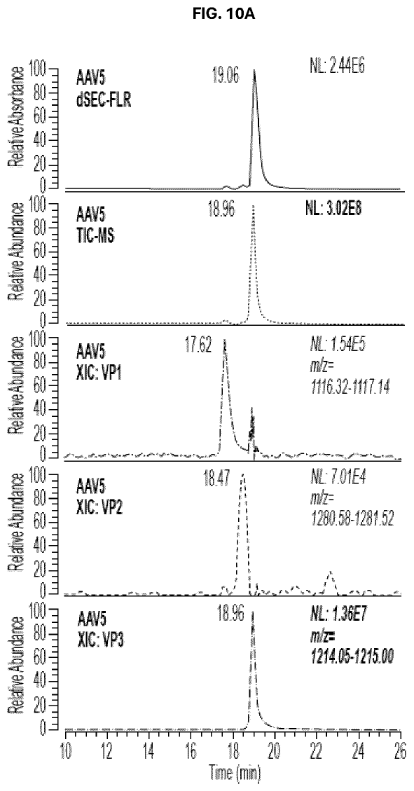

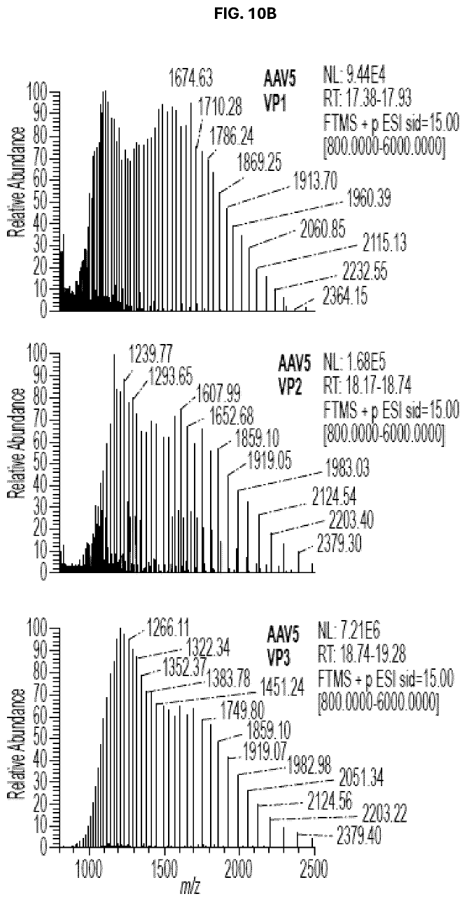

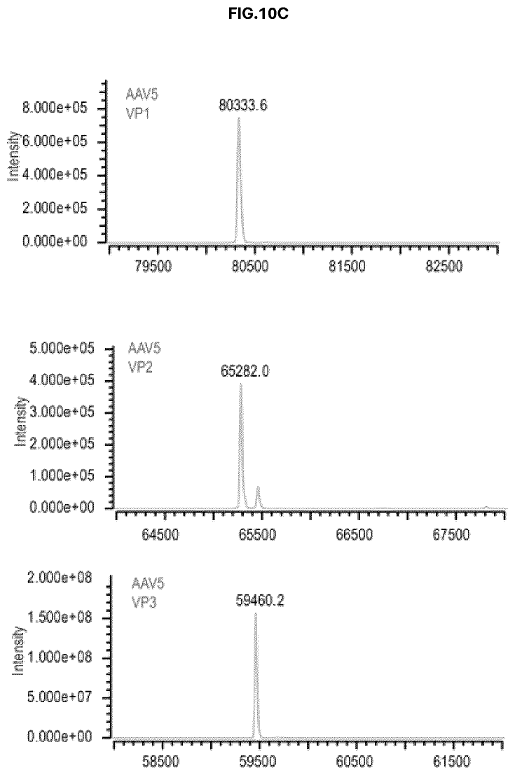

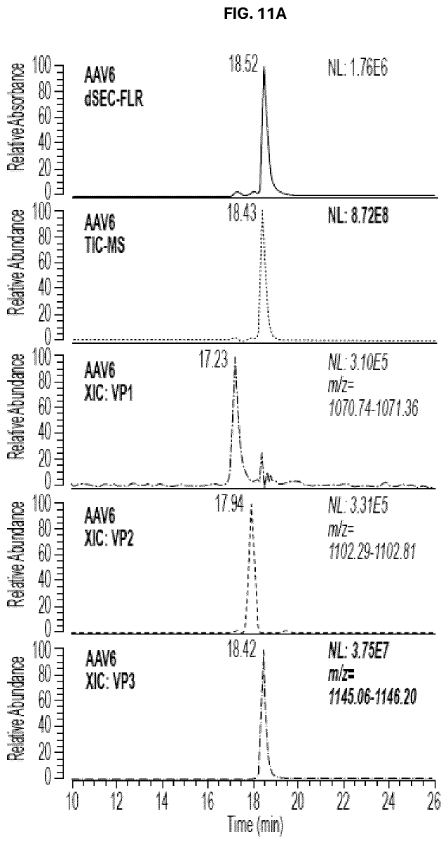

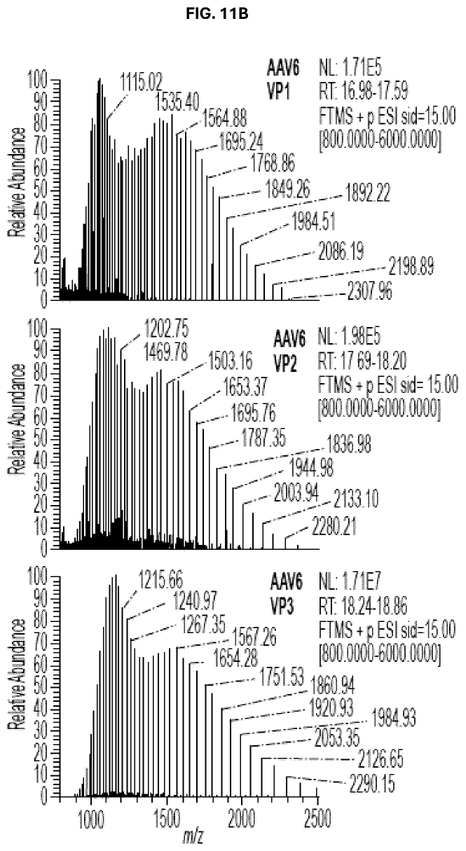

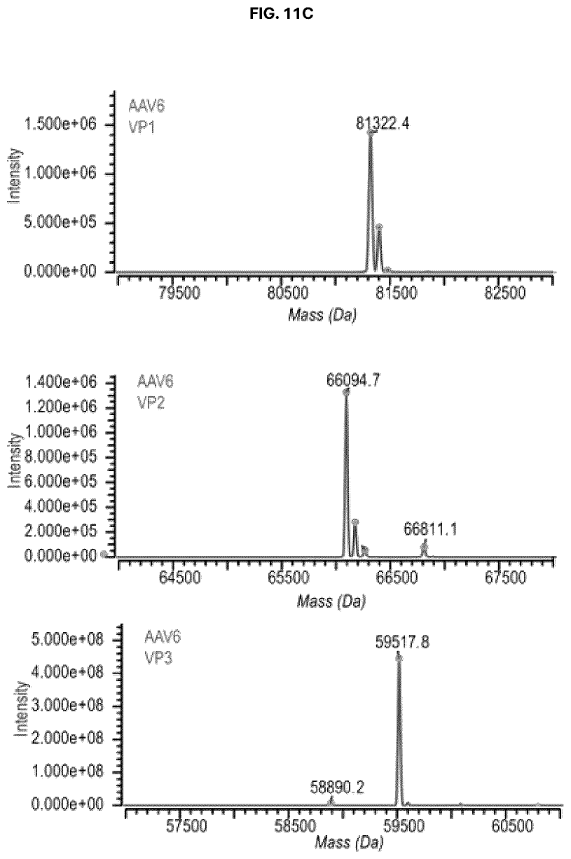

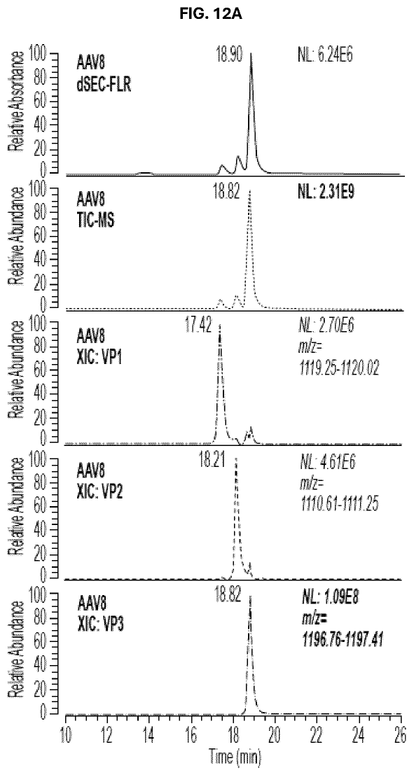

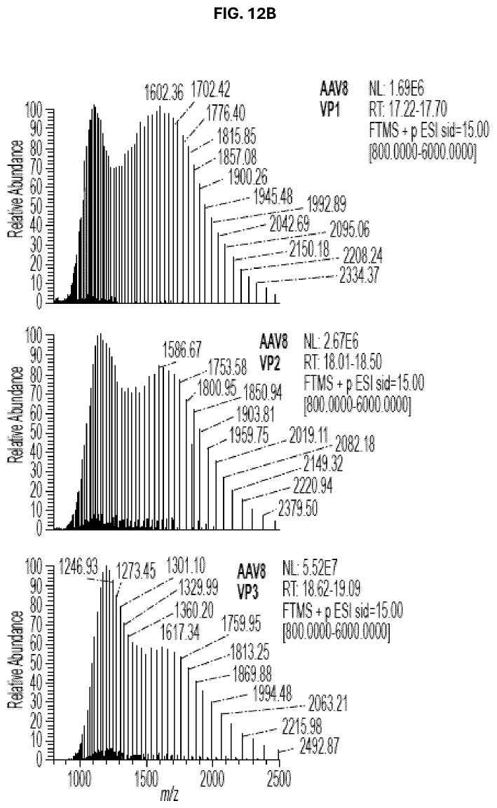

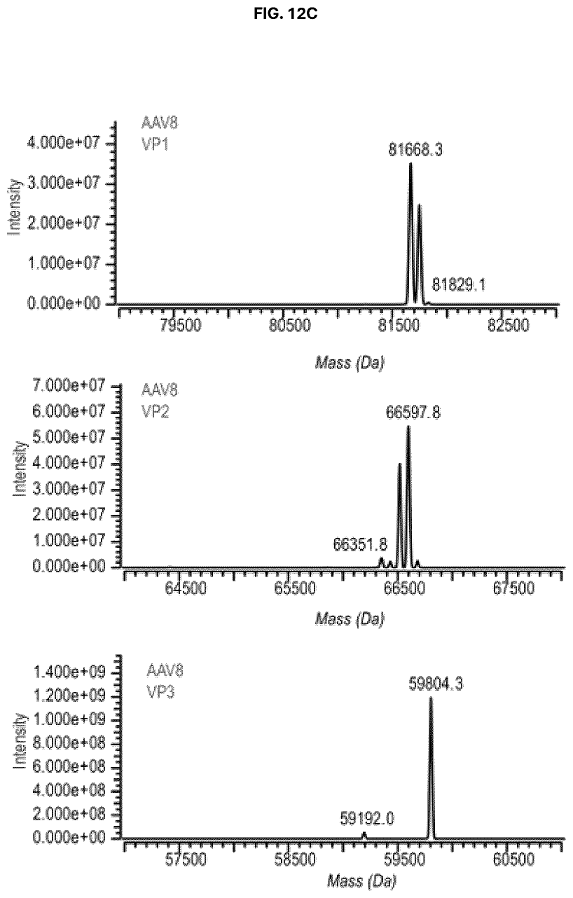

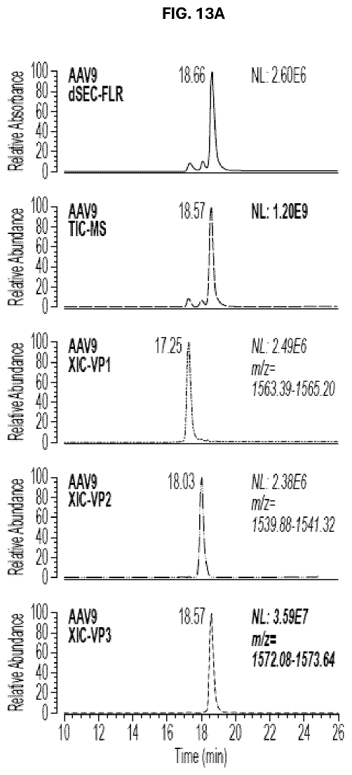

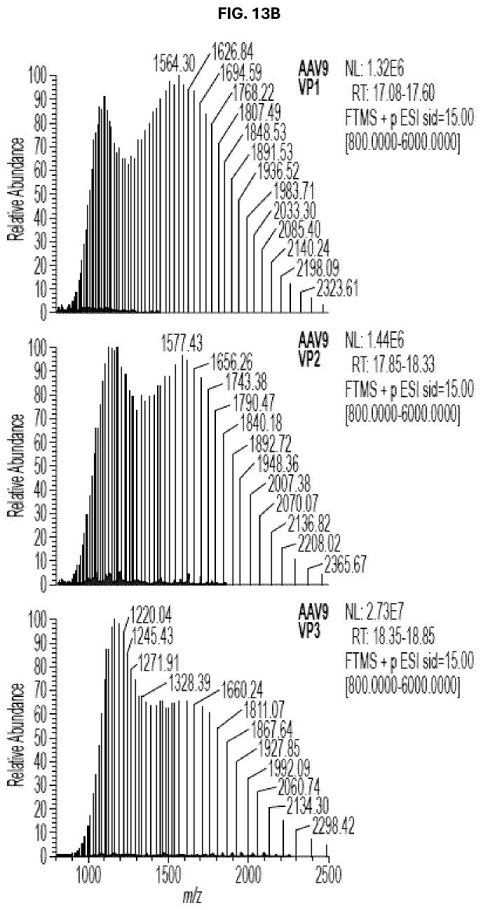

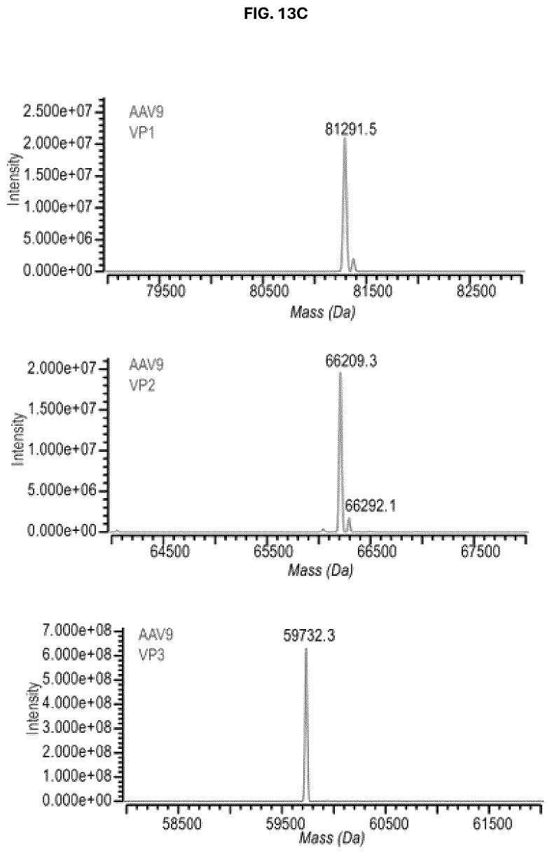

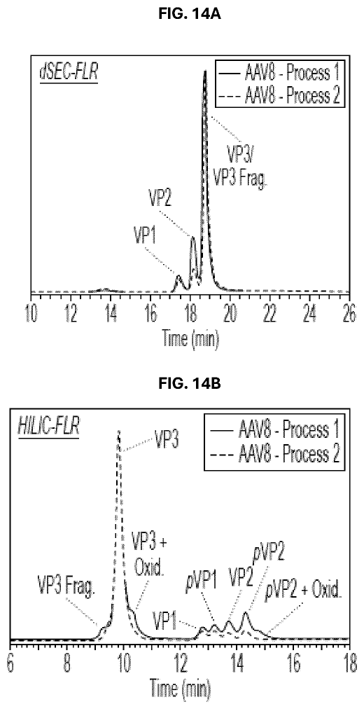

Proteoforms present at low abundances, such as bisphosphorylated VP1(Ac) and VP2, phosphorylated VP1(Ac), unphosphorylated and phosphorylated VP2 lacking N-terminal alanine and proline were also detected with high confidence (FIG. 7, see a, and Table 3). Notably, comparable mass spectral quality was obtained across all tested AAV samples, despite differences in serotype identity and VP(1-3) stoichiometry, allowing for facile identification of the heterogenous VP variants and AAV serotype confirmation (FIGS. 8-13 and Table 2). Given that formulated AAV samples are typically low concentration, sensitive and robust methods, such as dSEC, will be beneficial in providing detailed insights of product quality attributes using limited sample amounts (˜200 ng loading).

### Example 3. Comparison of AAV from Two Different Manufacturing Processes

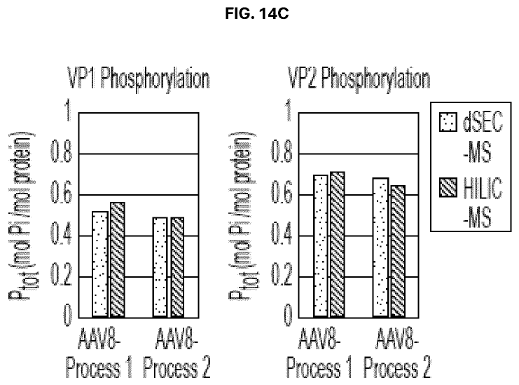

The utility of the dSEC-FLR-MS method was further demonstrated in a comparability case study, where two AAV8 lots produced from two different processes were compared at the intact capsid protein level. As demonstrated by the dSEC-FLR traces in FIG. 14A, distinct differences in the relative intensity of VP1 and VP2 (normalized against VP3) were observed between AAV8 produced using process 1 and that using process 2, where the former exhibited significantly higher levels of VP2 and slightly elevated levels of VP1 than the latter. To confirm this, the same samples were also subjected to HILIC-FLR-MS analysis, an established method for quantifying capsid stoichiometry and assessing capsid protein PTMs. Recapitulating the observations from the dSEC-FLR analyses, HILIC also detected higher relative levels of VP1 and VP2 in Process 1-generated compared to Process 2-generated AAV8 samples.

By integrating the FLR peak areas associated with each respective capsid protein, their relative abundances and the VP stoichiometry were quantified from both dSEC and HILIC analyses. As detailed in Table 4, the FLR measured relative abundances of each VP were highly comparable between dSEC and HILIC methods, displaying differences of less than 3% in relative-% FLR quantitation for all capsid proteins. As a result, the VP1:VP2:VP3 stoichiometry was determined to be 1.0:2.4:10.4 (by dSEC) or 1.0:2.4:8.9 (by HILIC) for the Process 1 sample, and 1.0:1.7:14.7 (by dSEC) or 1.0:1.2:12.4 (by HILIC) for the Process 2 sample, respectively. These results confirmed the different stoichiometry of AAV8 samples produced by these two processes, where process 2 resulted in fewer copies of VP1 and VP2 than process 1 (Table 4).

TABLE 4
Comparison of FLR-based quantitation of AAV8 Process 1 and
Process 2 samples analyzed by HILIC-FLR and dSEC-FLR.

| | | VP1 | VP2 | VP3 | | |
|---|---|---|---|---|---|---|
| | Analytical | Rel. Abundance | | | Normalized | Avg. Capsid |
| Sample ID | Method | (%-Peak Area) | | | VP(1-3) Ratio | VP(1-3) Ratio |
| AAV8 | HILIC-FLR | 8.1% | 19.4% | 72.5% | 1.0:2.4:8.9 | 4.9:11.6:43.5 |
| Process 1 | dSEC-FLR | 7.2% | 17.7% | 75.1% | 1.0:2.4:10.4 | 4.3:10.6:45.0 |
| AAV8 | HILIC-FLR | 6.9% | 8.0% | 85.1% | 1.0:1.2:12.4 | 4.1:4.8:51.1 |
| Process 2 | dSEC-FLR | 5.8% | 9.6% | 84.6% | 1.0:1.7:14.7 | 3.5:5.7:50.8 |

In addition to VP stoichiometry characterization, the individual VP proteoform heterogeneity was also characterized by both dSEC and HILIC methods using online MS detection. In contrast to dSEC which can only chromatographically resolve capsid proteins into VP1, VP2, and VP3 populations, HILIC separation is also sensitive to protein truncation and PTMs such as phosphorylation and/or oxidation, resulting in several FLR peak features as denoted in FIG. 14B. Despite the differences in separation profiles, both methods successfully identified similar VP variants from both AAV8 process samples by MS detection. The variants primarily consisted of VP3(Ac), VP2, pVP2, VP1(Ac) and pVP1(Ac).

As mono-phosphorylation does not appreciably affect electrospray ionization or MS detection efficiency of the intact proteins, the relative distribution of the phosphorylated and unphosphorylated variants associated with each VP was estimated using their corresponding MS intensity. As expected, comparable quantitative data were obtained from both dSEC- and HILIC-MS, which showed higher phosphorylation levels for VP2 than VP1 (Table 5, FIG. 14C). Furthermore, both methods recapitulated similar quantitative trends between process 1 and process 2 samples, displaying slightly higher overall phosphorylation level in the former than the latter (FIG. 14C).

TABLE 5
Relative quantitation of VP1 and VP2 phosphorylation obtained
from HILIC-MS and dSEC-MS from AAV8 Process 1 and Process 2.

| | |
|---|---|
| HILIC-MS* | dSEC-MS |

| | | |
|---|---|---|
| | | Rel. Abundance |
| Sample ID | Proteoform ID | (%- MS Peak Area) |

| | | | |
|---|---|---|---|
| AAV8 | VP1 | 44.1% | 49.7% |
| Process 1 | pVP1 | 55.9% | 50.3% |
| | VP2 | 29.5% | 31.5% |
| | pVP2 | 70.5% | 68.5% |
| AAV8 | VP1 | 50.8% | 51.4% |
| Process 2 | pVP1 | 49.2% | 48.6% |
| | VP2 | 35.8% | 33.8% |
| | pVP2 | 64.2% | 66.2% |

*denotes that oxidized proteoforms of non-phosphorylated and monophosphorylated VP1 and VP2 detected by HILIC-MS were combined with their non-oxidized counterparts for relative quantitation of phosphorylation.

Both methods also successfully identified low levels of protein backbone truncated species, such as a N-terminal VP3 fragment present in AAV8. The N-terminal VP3 fragment was identified as removal of N-terminal Met212 and the subsequent acetylation of the following alanine rather than the canonical initiator Met204 site, presumably due to leaky scanning at the first initial codon of VP3. dSEC-MS was unable to successfully detect the oxidation-associated VP(1-2) variants that were chromatographically resolved and detected by HILIC-MS. This limitation is attributed to the difficulty of fully resolving the mass of low levels of oxidation variant (+16 Da) from that of the main species, particularly when the main species is a relatively larger protein, such as intact VPs (>50 kDa). Nevertheless, the use of dSEC for VP analysis can simplify FLR features for easier quantitation of VP stoichiometry while maintaining the ability to monitor common PTMs such as phosphorylation, which can affect the infectivity of AAVs.

### Example 4. Differential Detection of Empty and Full AAV Capsids

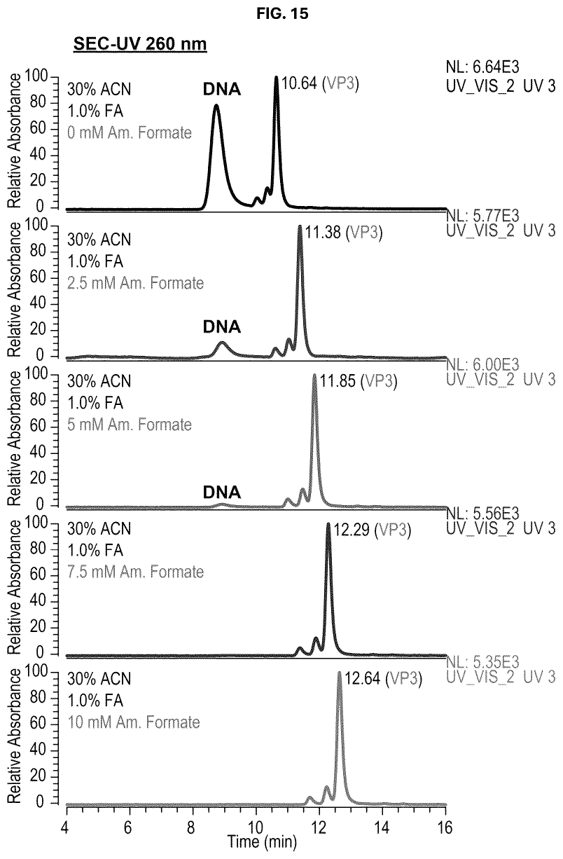

In addition to VP(1-3)-associated quality attributes, DNA content is another attribute to monitor during the development of AAV-based therapeutics. As AAV DNA payload molecules are significantly larger in hydrodynamic radii than capsid proteins, the dSEC method can be a promising tool to distinguish between DNA and VPs during AAV composition analysis. Considering the unique biophysical properties of DNA molecules, the dSEC mobile phase condition was reevaluated and optimized to a composition of 30% ACN and 1% FA, which provided best on-column recovery of DNA (FIG. 15). Given the limitations of FLR detection for DNA due to the weak intrinsic fluorescence from nucleic acids and nucleotides (quantum yield (Φ)=10⁻⁵ to 10⁻⁴ compared to Φ=10⁻¹ for tryptophan [41]), a ultraviolet (UV) detector was added in tandem to FLR post dSEC separation (dSEC-FLR/UV). This setup allows for multiple wavelength UV monitoring at both 260 nm and 280 nm, corresponding to the absorbance maxima for DNA and proteins, respectively, for the calculation of UV260/280 ratios to aid in chromatography peak identifications.

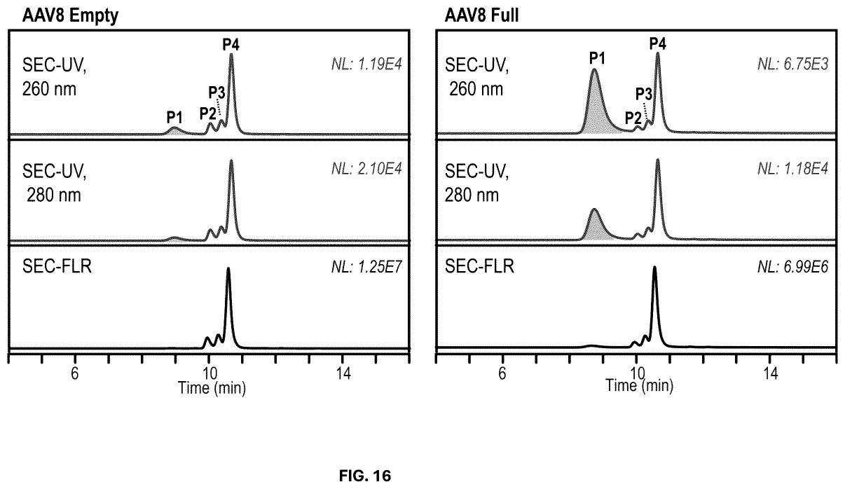

Using a pair of empty and full AAV8 samples with various encapsulated DNA payload, the DNA molecules were efficiently resolved from the capsid proteins (FIG. 16). The DNA molecule eluted as a broad peak under UV detection prior to VP1. In addition to the expected elution order, this DNA peak (P1) assignment was also supported by its UV260/280 absorbance ratio, which was consistently measured at about 1.2 in both the full and empty sample (Table 6). In contrast, the measured UV260/280 ratios for VP1, VP2, and VP3 peaks (P2-P4) from both samples were all approximately 0.6, which were in line with expectations that proteins exhibit higher UV absorbance at 280 nm than 260 nm.

TABLE 6
Identification and assignment of dSEC-FLR/UV peak
features from AAV8 full and empty capsids, based
on UV260/UV280 peak height quantitation.

UV 260/280 Ratio

| Elution | | AAV8 | AAV8 |
|---|---|---|---|
| Order | Assignment | Empty | Full |
| P1 | DNA | 1.20 | 1.19 |
| P2 | VP1 | 0.57 | 0.65 |
| P3 | VP2 | 0.57 | 0.60 |
| P4 | VP3 | 0.57 | 0.57 |

Furthermore, the relative abundance of DNA molecules (P1) in the AAV8 full sample was approximately 10× higher than the AAV8 empty sample, when normalized against VP(1-3) (P2-P4) using the UV signal (Table 7). This shows the presence of substantially greater encapsulated DNA payload in the AAV8 full sample than the AAV8 empty sample. Finally, the FLR detection of P1 in both samples exhibited a weak signal (less than 1% relative FLR-intensity), further supporting its oligonucleotide nature. Taken together, these findings show the capability of dSEC to effectively separate and identify DNA and protein contents in AAV samples, and its potential applicability in enabling semi-quantitative DNA percentage comparisons between different AAV production lots.

TABLE 7
Experimental UV 260 and 280 nm absorbance values for P1-P4, and the UV-based
quantitation of DNA normalized against VP(1-3) in empty and full AAV samples.

| | | AAV8 Empty | | | AAV8 Full | | |
|---|---|---|---|---|---|---|---|
| | | UV 260 | UV 280 | UV 260 | UV 260 | UV 280 | UV 260 |
| | | nm (Peak | nm (Peak | (DNA/ | nm (Peak | nm (Peak | (DNA/ |
| Elution | | Height, | Height, | VP(1-3) | Height, | Height, | VP(1-3) |
| Order | Assignment | mAU) | mAU) | Ratio | mAU) | mAU) | Ratio |
| P1 | DNA | 1001 | 836 | 0.063 | 5359 | 4506 | 0.63 |
| P2 | VP1 | 1677 | 2920 | | 592 | 914 | |
| P3 | VP2 | 2103 | 3678 | | 1090 | 1819 | |
| P4 | VP3 | 12120 | 21300 | | 6859 | 11970 | |

The acidic conditions of the mobile phase (approximately pH 2-3) can lead to an underestimation of the UV 260/280 ratio for oligonucleotides, potentially accounting for the lower-than-anticipated UV 260/280 ratio observed for P1. Furthermore, increasing the concentration of ammonium (NH₄⁺) additive (from 0-10 mM) led to a decrease in on-column recovery of DNA. For example, when the AAV8 full sample was analyzed using a mobile phase with 7.5 mM ammonium formate, the UV peak at 260 nm corresponding to the DNA molecule could not be detected at the expected elution window (FIG. 15).

Additionally, oligonucleotides may suffer from non-specific adsorption when working at lower pH ranges due to possible ionic interactions with electropositive metal-oxide surfaces, such as chromatography lines and column hardware. Further, the adoption of a SEC column fabricated with hydrophilic surface hardware reduced unwanted secondary interactions.

With the rapid growth of AAV-based gene therapies in the pharmaceutical market, there is an increased demand for improved analytical methods to support product and process development. In the present disclosure, a robust and effective SEC-based method was developed that enables superior size-based separation of VP components and can be broadly applied to a variety of AAV serotypes. With simultaneous detection by FLR, UV, and MS, this method was demonstrated to be highly sensitive and suitable for AAV serotype identification, stoichiometric assessment, and PTM/truncation variant analysis. This method also enabled semi-quantitative analysis of DNA molecules from AAV capsids, which can be readily applied to compare the relative DNA contents of different AAV lots. Additionally, due to its separation mechanism and robust detection sensitivity, this method also provided characterization of engineered AAV capsids, as well as other complex AAV-based modalities, such as retargeting AAVs with affinity ligands fused to the VPs.

### Example 4. Denaturing SEC-MS for Characterization of Low Molecular Weight Variants in Antibodies

Low molecular weight species (LMW) species are CQAs (critical quality attributes) to be monitored in pharmaceutical products. The present study analyzed whether dSEC could be applied towards effective LMW detection of mAb species from both IgG1, IgG4, and bispecific molecules. It was hypothesized that a larger SEC stationary pore size, which increased the denaturing SEC resolution of AAV viral proteins which range from (˜60-80 kDa), might be amenable to LMW analysis for mAb molecules, and facilitate LMW identification.

The study demonstrates that dSEC provides high sensitivity of these LMW species, while enabling simplified grouping of LMW species into specific classes for facilitated identification.

dSEC can be effectively applied to both IgG1 (kappa and lambda molecules), IgG4 and bispecific antibodies, and can also be used to analyze molecules subjected to thermal stress conditions, revealing different LMW pathway formations that are common in IgG1 and IgG4 molecules.

This study demonstrates that under the selected denaturing conditions, a large pore size such as a 450 A column are effective for LMW analysis, due to the increased denaturation and larger hydrodynamic radii and unfolding which occurs under denaturing conditions. This method is effective for even the analysis of smaller LMW species (5-25 kDa), such as VH, CH3, or free LC w/modifications-indeed, 450 A appeared to have sufficient resolving power for these smaller observed fragments under denaturing conditions, without co-eluting into the lower MW region where salt/non-volatile sample components usually elute during SEC.

This study demonstrates that using this mobile phase condition also provides enhanced sensitivity for HMW species, while maintaining strong separation. These conditions can help with understanding pathway formation of LMW species under various stability conditions (thermal, photo, pH, oxidative stress), and facilitate understanding the formation of observed LMWs in pharmaceutical products.

Comparison of Limited Inter-Chain Reduced mAb-1 Denaturing SEC Separation with Different Columns and Mobile Phase Compositions.

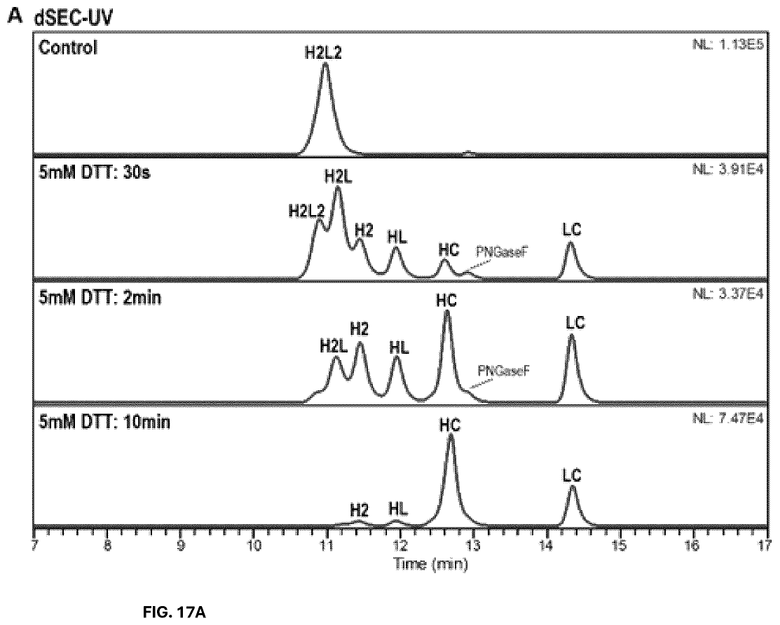

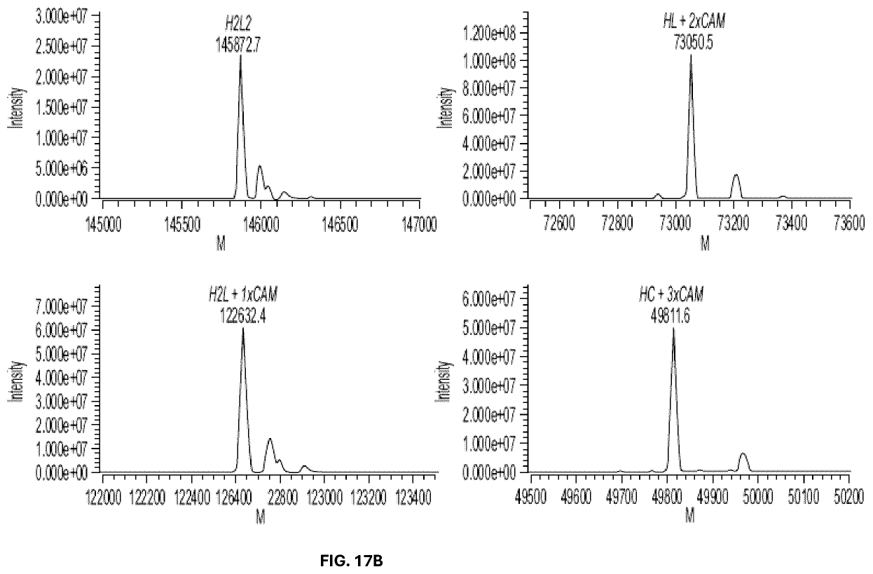

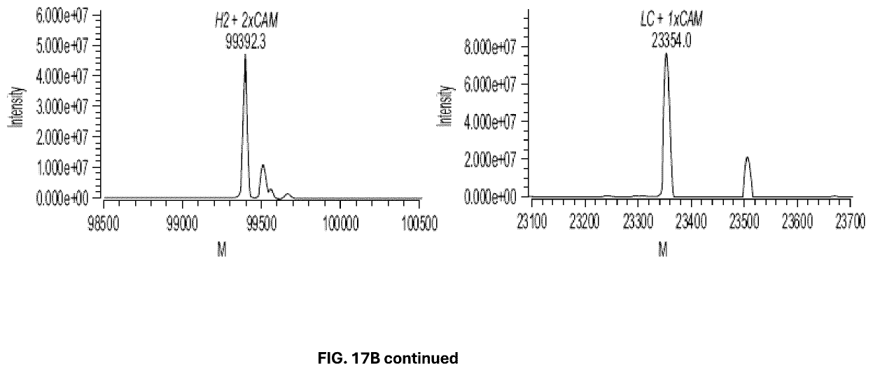

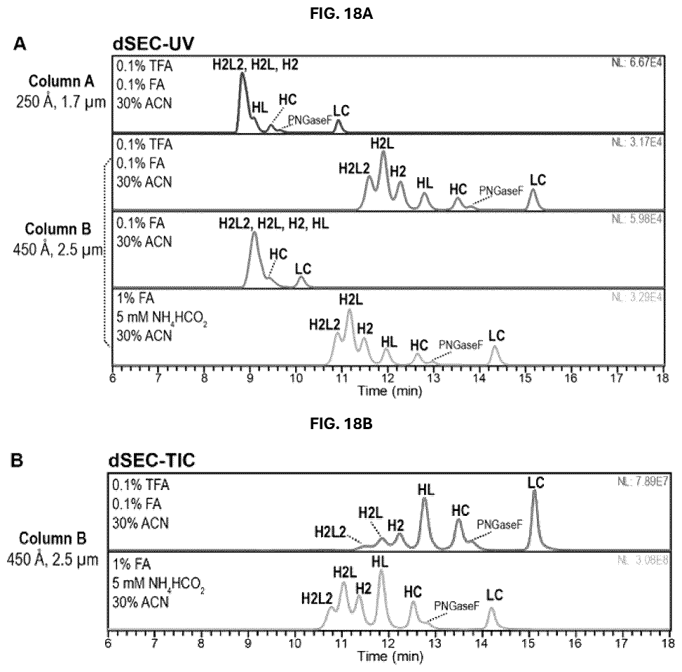

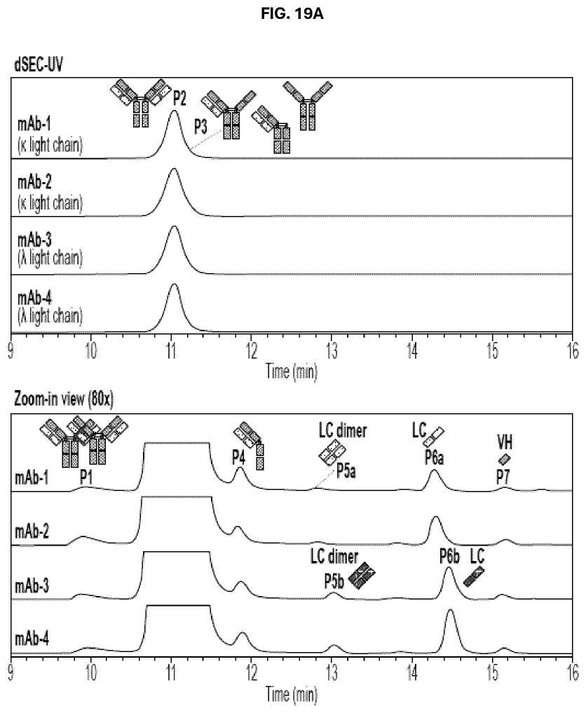

To evaluate denaturing SEC for the analysis of low molecular weight species (LMWs) using a wide-pore size SEC column (450 Å), we generated mAb size-variants by performing disulfide bond reduction using 5 mM DTT at 25° C. at various timepoints (30 s, 2 min, 10 min) on a model mAb treated with PNGase F to remove N-linked glycosylation and simplify mass spectral heterogeneity (FIG. 17A). The use of 5 mM DTT is a mild reduction condition which is intended to partially disrupt the most solvent-accessible mAb disulfide bonds, such as the interchain disulfide bonds that pair between the two heavy chain molecules, and heavy chain-light chain molecules. Following this incubation with DTT, the reduction was quenched by the addition of 15 mM IAA to artificially generate LMW species, which include mAb missing light chain (H2L), heavy chain dimer (H2), half molecule (HL), heavy chain (HC), light chain (LC), in addition to incompletely reduced mAb (H2L2). These LMW identities were confirmed based on the observed mass shift in accordance with the expected number of reduced and CAM-alkylated interchain cysteine residues of each species, as determined by accurate intact mass measurement (FIG. 17B). As demonstrated in FIG. 17A, the wide pore size (450 Å) SEC column exhibited strong separation of artificially generated mAb LMW species less than ˜ 100 kDa and suggested further examination for the application of a widepore SEC column for LMW analysis under denaturing conditions.

Comparison of Limited Inter-Chain Reduced mAb-1 Denaturing SEC Separation with Different Columns and Mobile Phase Compositions.

Two different columns were evaluated, using a 0.1% TFA, 0.1% FA, and 30% ACN composition. We used the 200 Å, 1.7 μm condition, for separation using small particle size and smaller pore size for efficient fractionation of LMW species less than 100 kDa. Using the same mobile phase conditions, we found that H2L2, H2L, H2 largely coeluted together, with increased separation between HC and LC. However, using Column B, which has a larger pore size, we found that we had later elution of all of the mAb-a protein standards, along with improved separation between H2L2, H2L, and H2, along with separation of HL, HC, and LC. Based on this evaluation, we used the 450 Å column, for further optimization and analysis.

We then compared different mobile phase parameters, including 0.1% TFA, 0.1% FA and 30% ACN, with the optimized mobile phase which was used for analysis of AAV VP(1-3). We found that the 0.1% FA, 30% ACN condition resulted in relatively very poor resolution, which suggested that 0.1% TFA was required as an ion-pairing reagent for denaturing SEC separation. Interestingly, the 0.1% TFA, 0.1% FA, 30% ACN condition demonstrated comparable performance to the 1% FA, 5 mM ammonium formate, and 30% ACN condition. However, when comparing the effect of mobile phase composition on TIC-MS intensity, the mobile phase with TFA resulted in ion suppression of the larger LMW species such as H2L and H2 compared to the 1% FA, 5 mM Am. formate condition. These observations suggested the use of the widepore SEC column with 1% FA, 5 mM ammonium formate, and 30% ACN for the analysis of LMW in various mAb formats.

### Denaturing SEC-MS Reveals Favored LMW Pathways in IgG1 Kappa and Lambda LC Molecules.

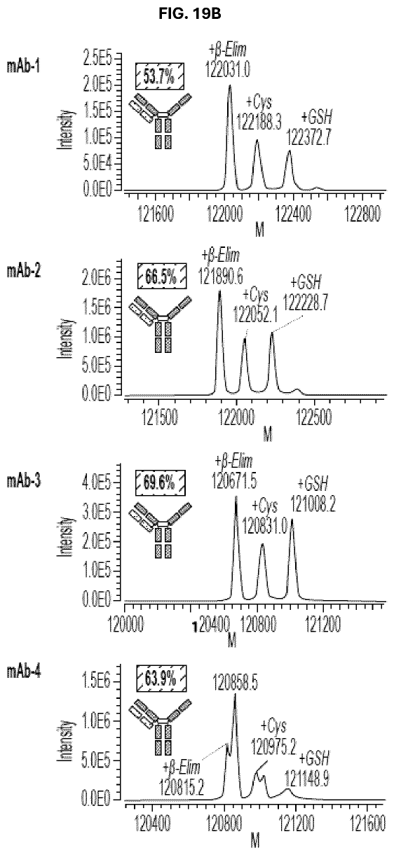

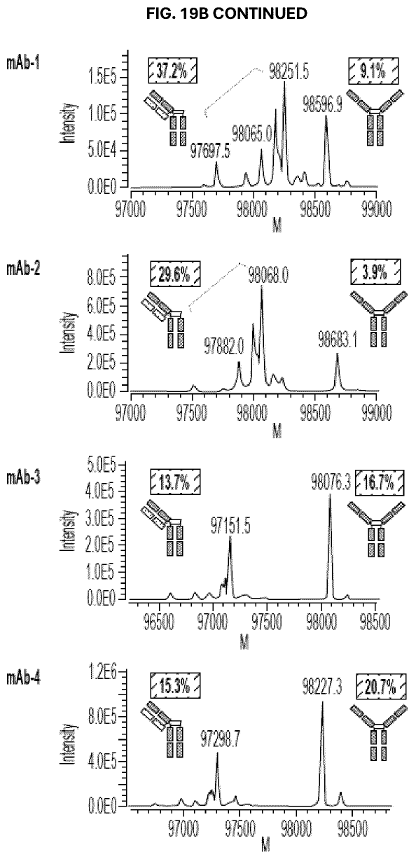

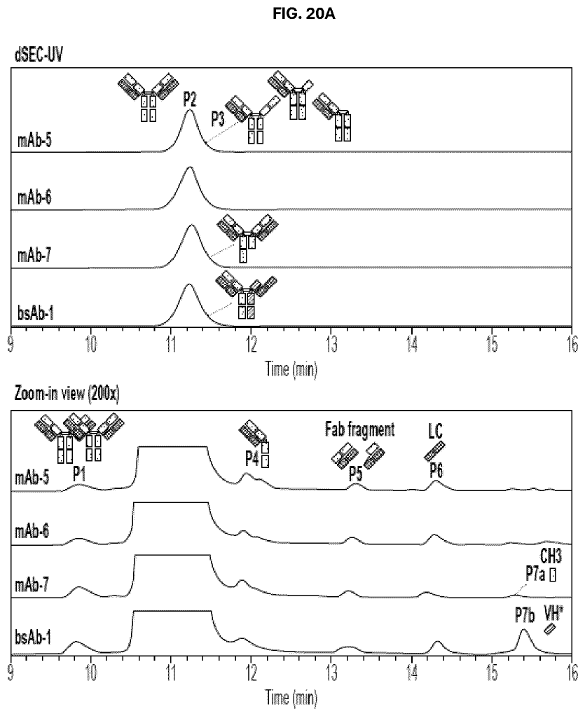

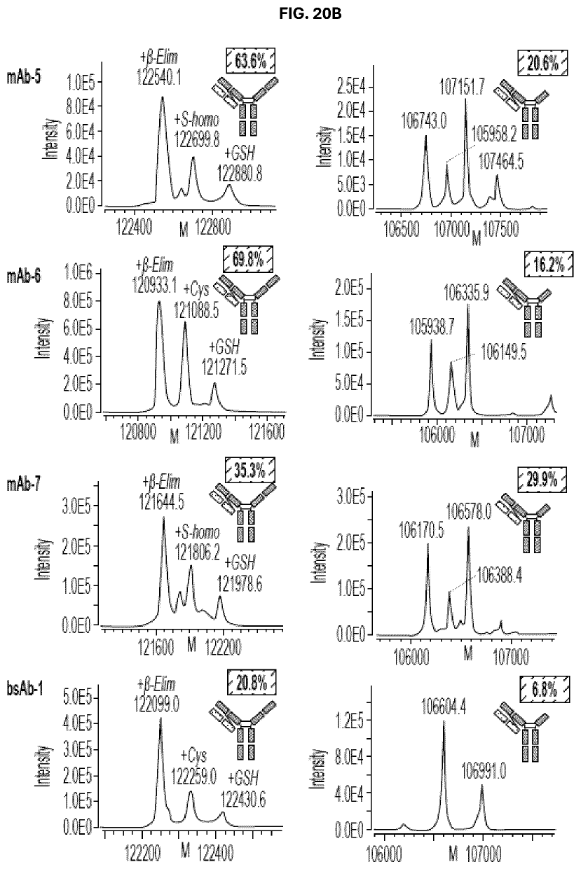

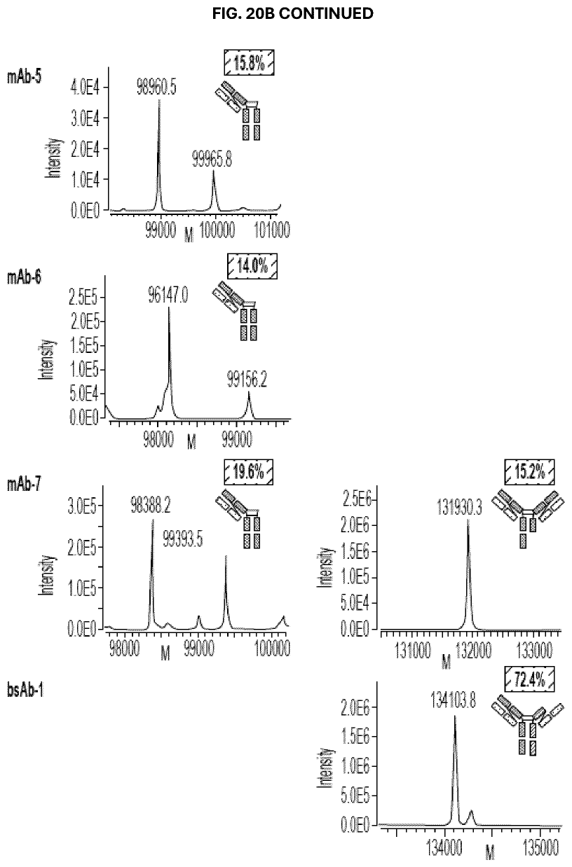

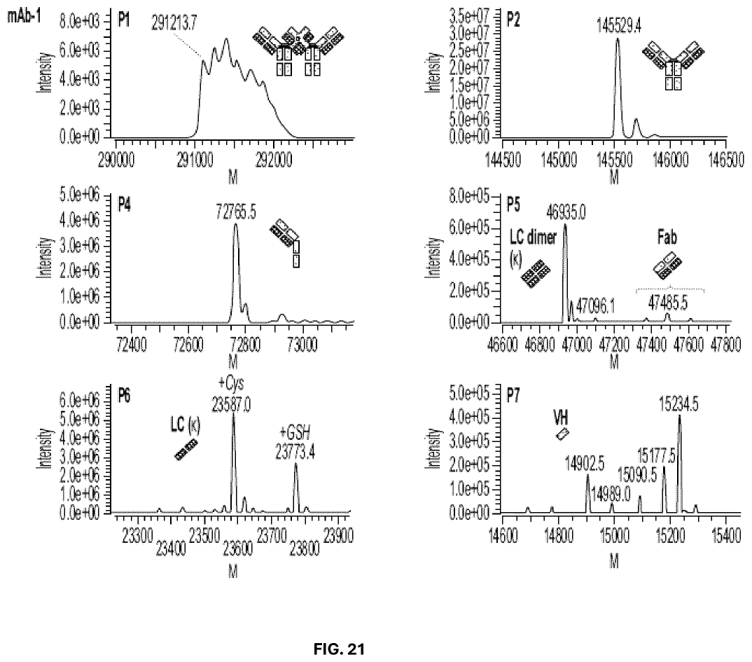

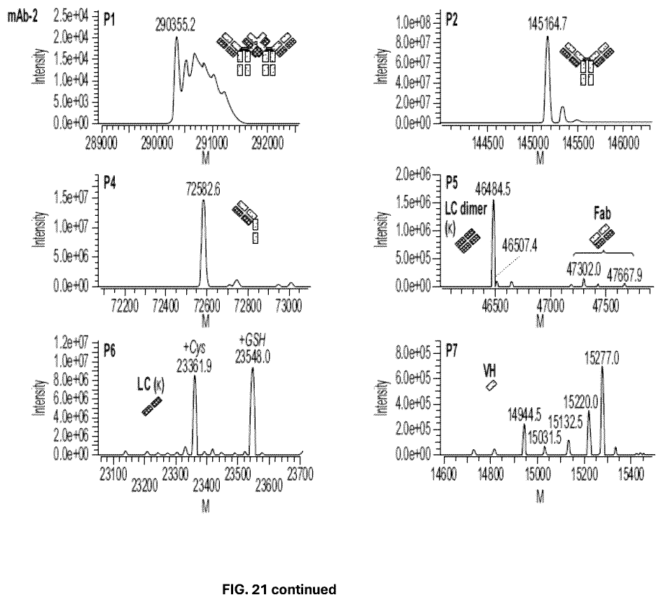

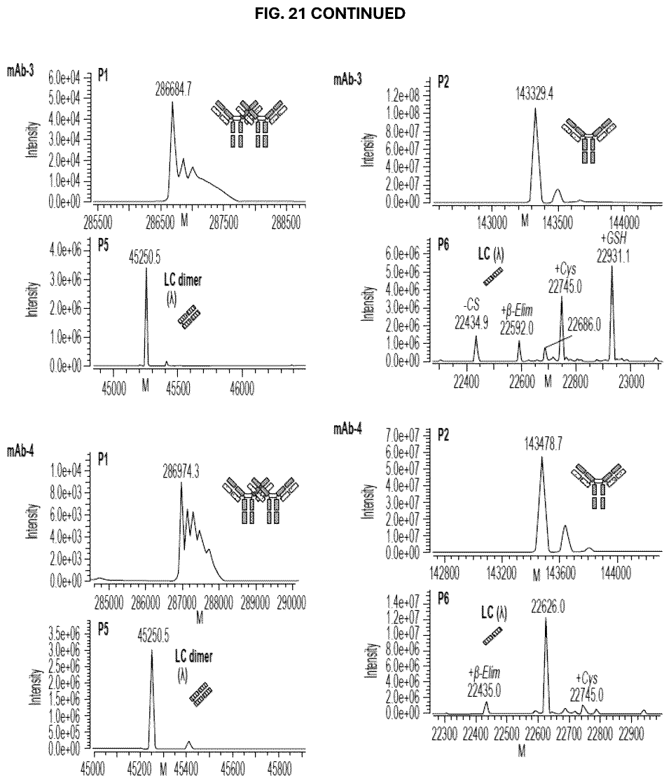

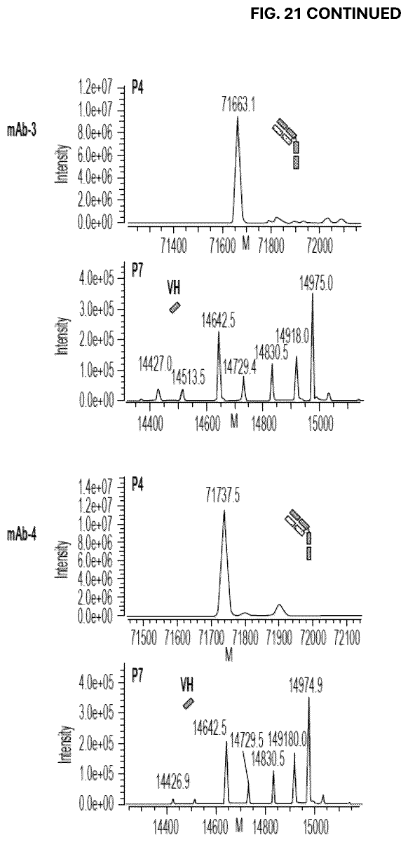

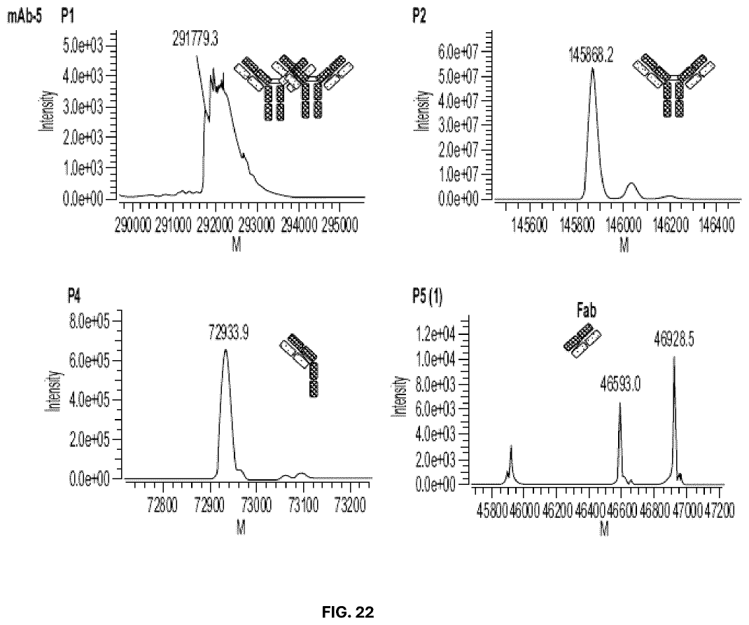

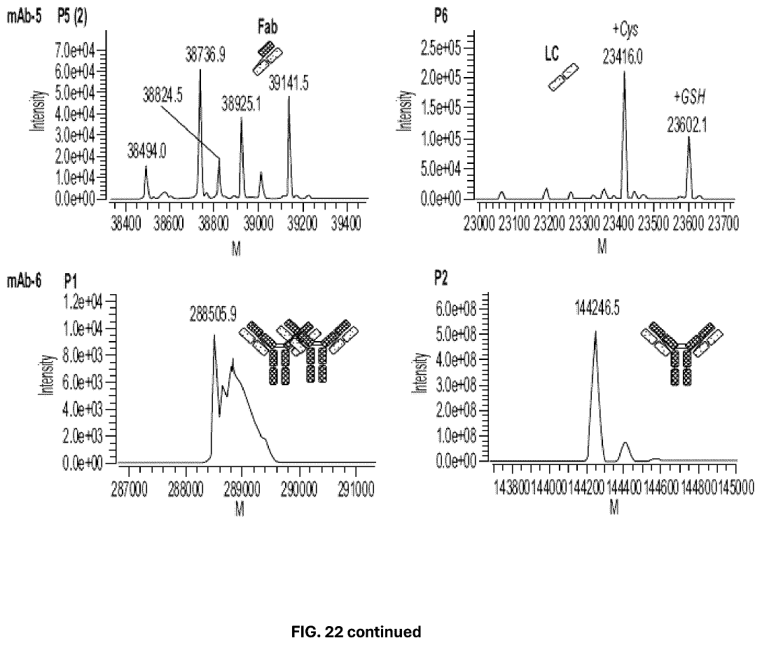

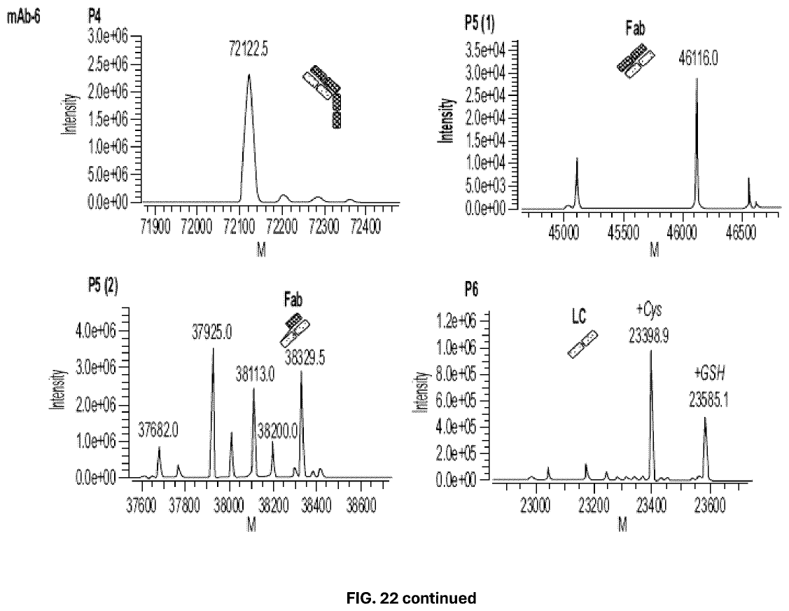

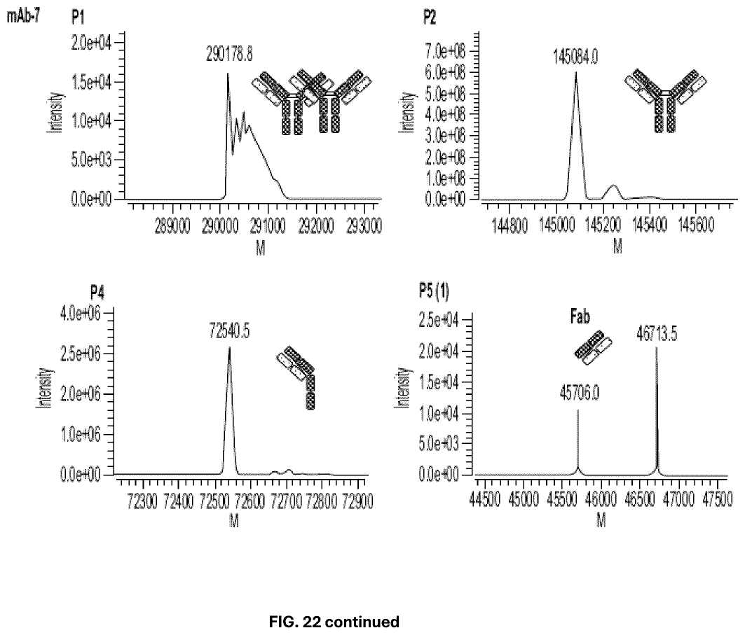

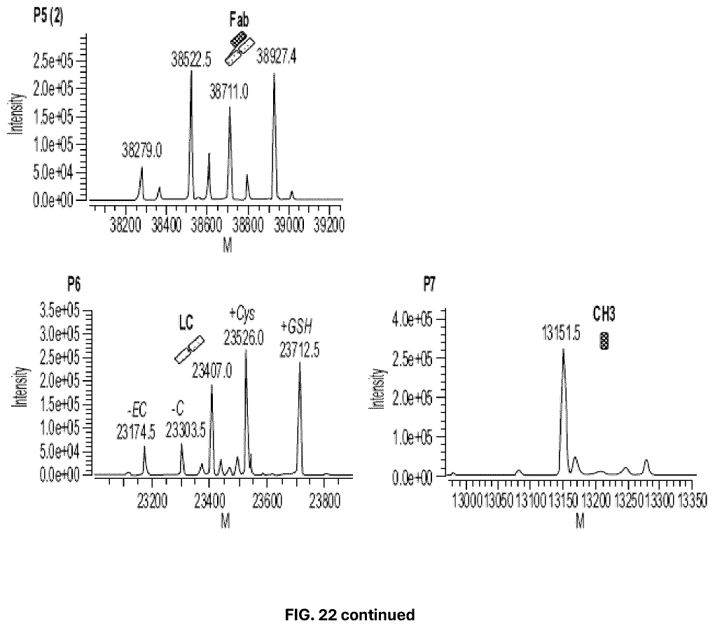

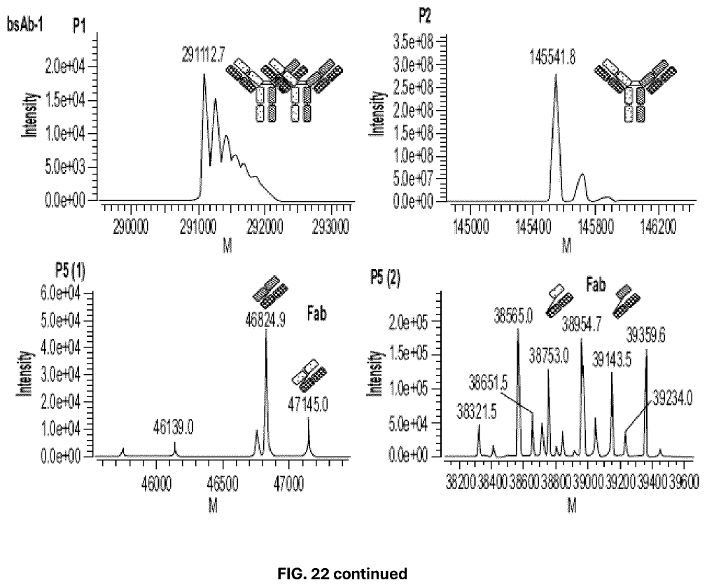

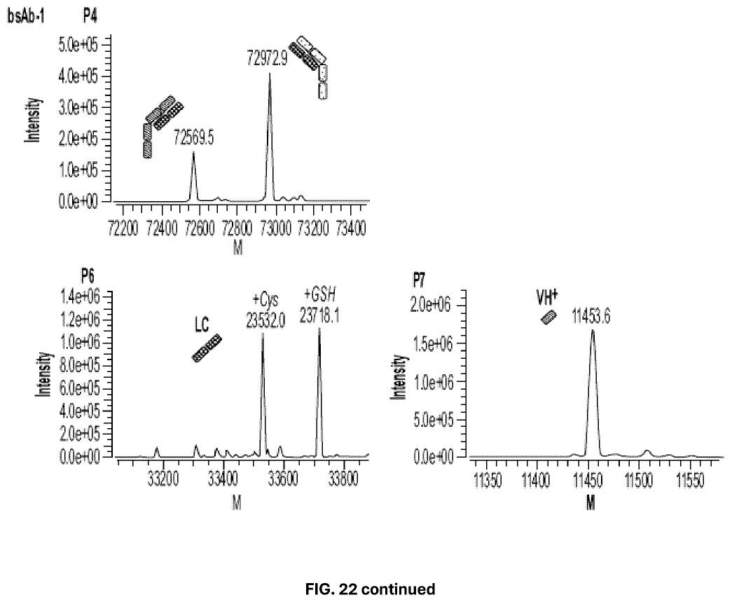

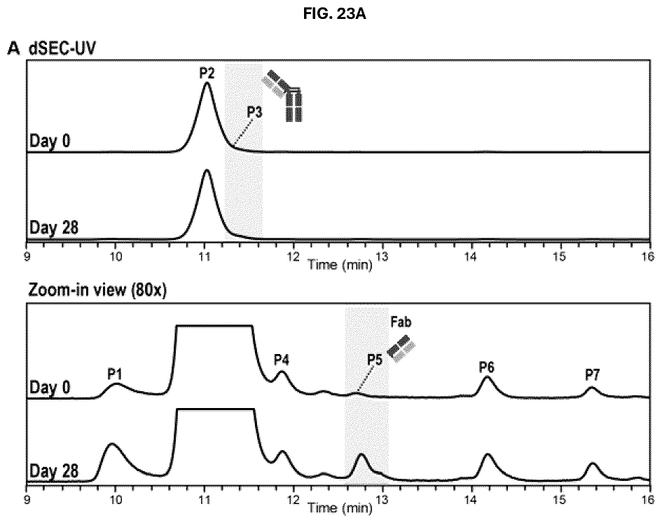

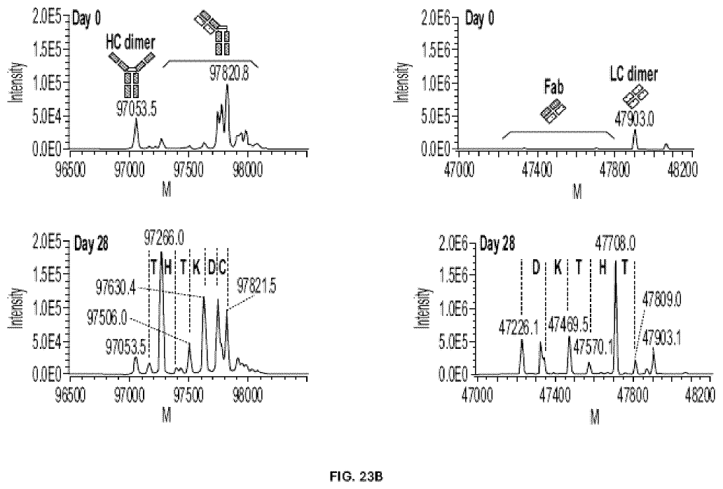

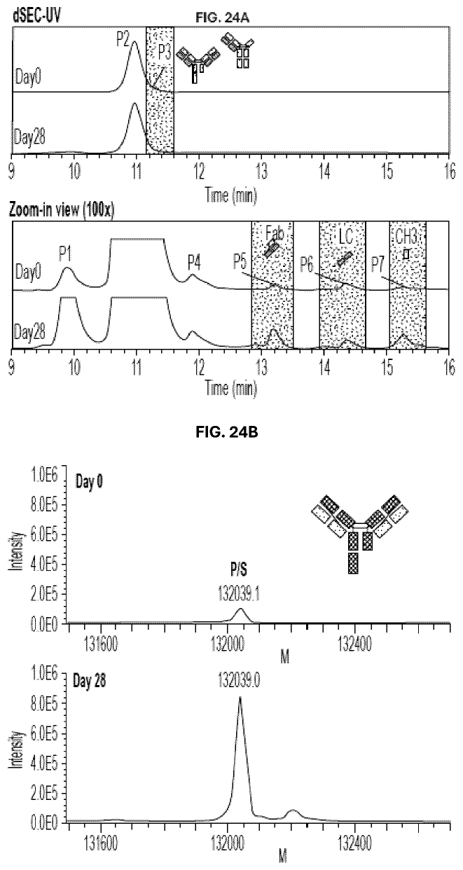

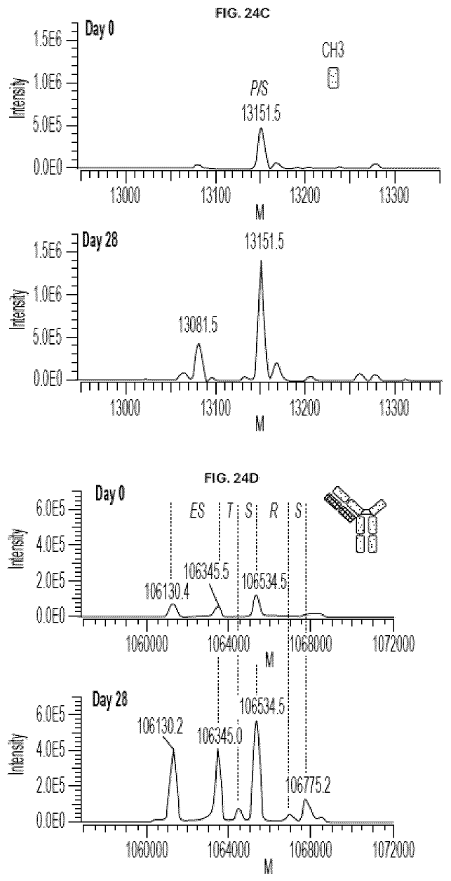

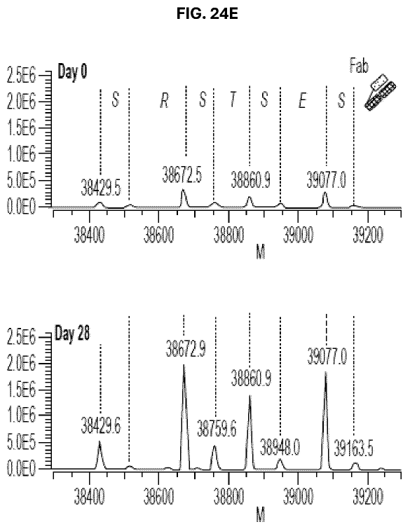

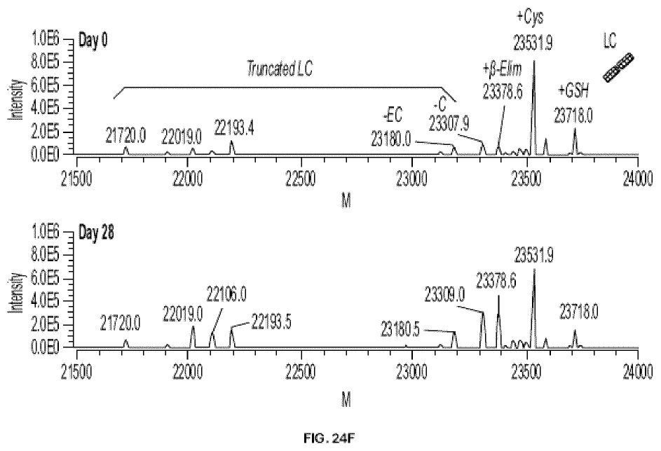

The dSEC-MS parameters and method were applied to survey four different deglycosylated IgG1 mAbs featuring either Kappa or Lambda light chain isotypes to assess the applicability of this dSEC method to assess LMW formation in these formats. The LC isotype can affect the stability and function of human IgG1. Due to the unique positioning of the CL-CHI interchain disulfide at the end of the CH1 and near the beginning of the hinge region, rather than at the beginning of the CH1 domain in other IgG subclasses, the position of the disulfide influences the mechanism of disulfide-bond breakage leading to the formation of unique LMW species. The representative dSEC-UV separation of IgG1 LMW species is demonstrated in FIG. 19A, with an enlarged view of the baseline at 80× magnitude to visualize the excellent resolution of several UV peaks corresponding to LMW species such as HL (P4), LC dimer (P5a/P5b), LC (P6a/P6b), and VH domain fragments (P7). While the dSEC-UV chromatograms appear visually similar across the four molecules due to the size-based separation mechanism of the LMW species, the LMWs arising from lambda and kappa LC isotype mAbs have different retention times and different propensities towards their formation. As demonstrated by the deconvoluted mass spectra corresponding to the elution window of each dSEC-UV peak, the intact MS data provides detailed insights into the mechanisms of LMW formation, such as H2L, which have different PTM profiles that may be related to cell metabolism conditions such as high cysteine feed or cellular glutathione levels, and may disintegrate interchain disulfide bonds during fermentation. MS can also show differences that are not observed by the LC profile. For example, H2L modifications are consistent with the observed LC modifications, because these species are generated through the same cysteine related mechanisms.

While large LMW species such as H2L, mAb missing Fab arm, and HC dimer were not baseline separated from the main peak, they can still be accurately characterized as shown from the deconvoluted spectra shown in FIG. 19B. After deconvolution, a variety of cysteine-related PTMs such as beta-elimination, cysteinylation, and glutathionylation were observed in IgG1 mAbs featuring either Kappa or Lambda light chain. This was further confirmed by the deconvoluted MS spectra of LC, which also featured PTMs such as cysteinylation, GSH, and B elimination. However, formation of other large LMW species such as mAb missing Fab arm and HC dimer appeared to vary between kappa and lambda light chain mAbs, with HC dimer appearing to be a much higher proportion of large LMW species. This suggests that IgG1 mAbs featuring lambda LC may have a less stable disulfide bond between the LC and HC compared to that in IgG kappa.

### Denaturing SEC-MS Reveals Favored LMW Pathways in IgG4 Molecules.

A survey of four IgG4 molecules was performed, including 3 monospecific and 1 bispecific antibodies (FIGS. 20A and 20B).

All references cited herein, including U.S. patent and applications are incorporated by reference in their entirety. The present disclosure is not to be limited in terms to the particular examples described in this application, which are intended as illustrations of various aspects. Many modifications and variations can be made without departing from its spirit and scope, as will be apparent to those skilled in the art. Functionally equivalent methods within the scope of the disclosure, in addition to those enumerated herein, will be apparent to those skilled in the art from the foregoing descriptions. Such modifications and variations are intended to fall within the scope of the appended claims.

What is claimed is:

1. A method for separating protein components of a viral capsid of a viral vector, comprising:
subjecting a sample comprising a viral vector to size exclusion chromatography (SEC) column under a denaturing mobile phase to separate the protein components of the viral capsid of the viral vector.

2. The method of claim 1, wherein the viral vector is an adeno-associated virus (AAV) vector comprising a serotype selected from AAV1, AAV2, AAV3, AAV4, AAV5, AAV6, AAV7, AAV8, AAV9, AAV10, AAV11, AAV12, variants thereof, and combinations thereof.

3. The method of claim 1, wherein the protein components of the viral capsid comprise VP1, VP2, and VP3 capsid proteins.

4. The method of claim 3, further comprising determining the stoichiometry of the VP1, VP2, and VP3 capsid proteins by detecting the capsid proteins by fluorescence to determine the relative abundance of the capsid proteins, thereby determining the stoichiometry of the VP1, VP2, and VP3 capsid proteins.

5. The method of claim 1, wherein the denaturing mobile phase comprises acetonitrile, formic acid, and ammonium formate in water.

6. The method of claim 5, wherein the concentration of acetonitrile is about 30%.

7. The method of claim 5, wherein the concentration of formic acid is about 1%.

8. The method of claim 5, wherein the concentration of ammonium formate is between about 5 mM to about 10 mM.

9. The method of claim 1, wherein a flow rate of the mobile phase for the SEC is between about 0.125 mL/min and about 0.2 mL/min.

10. The method of claim 1, further comprising subjecting the protein components to mass spectrometry to identify the separated protein components.

11. A method of identifying and/or characterizing at least one low molecular weight (LMW) antibody species in a sample, comprising:
(a) subjecting the sample to size exclusion chromatography (SEC) column under a denaturing mobile phase to separate the LMW antibody species; and
(b) detecting the LMW antibody species by mass spectrometry to identify and/or characterize at least one LMW antibody species.

12. The method of claim 11, wherein the denaturing mobile phase comprises acetonitrile, formic acid, and ammonium formate in water.

13. The method of claim 12, wherein the concentration of acetonitrile is about 30%.

14. The method of claim 12, wherein the concentration of formic acid is about 1%.

15. The method of claim 12, wherein the concentration of ammonium formate is between about 5 mM to about 10 mM.

16. The method of claim 11, wherein a flow rate of the mobile phase for the SEC is between about 0.125 mL/min and about 0.2 mL/min.

17. The method of claim 11, wherein the SEC is coupled to a mass spectrometer.

18. The method of claim 17, wherein the mass spectrometer comprises a heated electrospray ionization source, an electrospray ionization source, a nano-electrospray ionization source, a microflow-nanospray electrospray ionization source, or a desorption electrospray ionization source.

19. The method of claim 17, wherein the mass spectrometer comprises a time-of-flight mass analyzer, a magnetic/electric sector mass analyzer, a quadrupole mass analyzer, or an orbitrap mass analyzer.
